(19) United States
(12) Patent Application Publication
(10) Pub. No.: US 2007/0191350 A1
(43) Pub. Date: Aug. 16, 2007

(54) Combinations comprising alpha-2-delta ligands

(75) Inventors: Mark J. Field, Sandwich (GB); Richard G. Williams, Sandwich (GB)

Correspondence Address: PFIZER INC, 150 EAST 42ND STREET, 5TH FLOOR - STOP 49, NEW YORK, NY, 10017-5612, US

(73) Assignee: Pifizer Inc., New York, NY (US)

(21) Appl. No.: 10/598,712

(22) PCT Filed: Feb. 24, 2005
(86) PCT No.: PCT/IB05/00510

§ 371 Date: Sep. 12, 2006

Related U.S. Application Data
(60) Provisional application No. 60/560,416, filed on Apr. 7, 2004.

(30) Foreign Application Priority Data
Mar. 8, 2004 (GB) 0405200.7

(51) Int. Cl. A61K 31/551; A61K 31/519; A61K 31/496; A61K 31/195

(52) U.S. Cl. 514/220; 514/259.41; 514/561; 514/364; 514/254.07

(57) ABSTRACT

The instant invention relates to a combination, particularly a synergistic combination, of an alpha-2-delta ligand and an atypical antipsychotic, and pharmaceutically acceptable salts thereof, pharmaceutical compositions thereof and their use in the treatment of pain, particularly neuropathic pain.

### FIELD OF THE INVENTION

This invention relates to a combination of an alpha-2-delta ligand and an atypical antipsychotic. The invention further relates to a combination of an alpha-2-delta ligand and an atypical antipsychotic for the treatment of pain. It also relates to a method for treating pain through the use of effective amounts of a combination of an alpha-2-delta ligand and an atypical antipsychotic. The invention further relates to a synergistic combination of an alpha-2-delta ligand and an atypical antipsychotic and the use of such for the treatment of pain.

### BACKGROUND TO THE INVENTION

An alpha-2-delta receptor ligand is any molecule which binds to any sub-type of the human calcium channel alpha-2-delta sub-unit. The calcium channel alpha-2-delta sub-unit comprises a number of receptor sub-types which have been described in the literature:
e.g. N. S. Gee, J. P. Brown, V. U. Dissanayake, J. Offord, R. Thurlow, and G. N. Woodruff, J-Biol-Chem 271 (10):5768-76, 1996, (type 1); Gong, J. Hang, W. Kohler, Z. Li, and T-Z. Su, J. Membr. Biol. 184 (1):35-43, 2001, (types 2 and 3); E. Marais, N. Klugbauer, and F. Hofmann, Mol. Pharmacol. 59 (5):1243-1248, 2001. (types 2 and 3); and N. Qin, S. Yagel, M. L. Momplaisir, E. E. Codd, and M. R. D'Andrea. Mol. Pharmacol. 62 (3):485-496, 2002, (type 4). They may also be known as GABA analogs.

Alpha-2-delta ligands have been described for the treatment of a number of indications. The best known alpha-2-delta ligand, gabapentin (Neurontin®), 1-(aminomethyl)-cyclohexylacetic acid, was first described in the patent literature in the patent family comprising U.S. Pat. No. 4,024,175. The compound is approved for the treatment of epilepsy and neuropathic pain.

A second alpha-2-delta ligand, pregabalin, (S)-(+)-4-amino-3-(2-methylpropyl)butanoic acid, is described in European patent application publication number EP641330 as an anti-convulsant treatment useful in the treatment of epilepsy and in EP0934061 for the treatment of pain.

Further alpha-2-delta ligands are described in the following documents.

International Patent Application Publication No. WO0128978, describes a series of novel bicyclic amino acids, their pharmaceutically acceptable salts, and their prodrugs of formula:

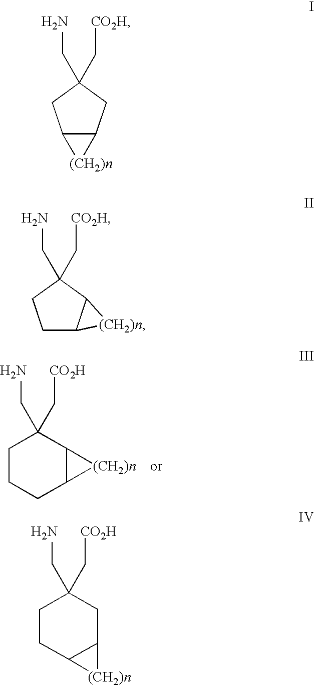

wherein n is an integer of from 1 to 4, where there are stereocentres, each center may be independently R or S, preferred compounds being those of Formulae I-IV above in which n is an integer of from 2 to 4.

International Patent Application No. WO2004/039367 describes compounds of the formula (I), below; 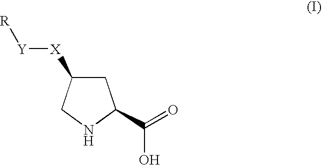
wherein
either X is O, S, NH or CH₂ and Y is CH₂ or a direct bond, or Y is O, S or NH and X is CH₂; and
R is a 3-12 membered cycloalkyl, 4-12 membered heterocycloalkyl, aryl or heteroaryl, where any ring may be optionally substituted with one or more substituents independently selected from
halogen, hydroxy, cyano, nitro, amino, hydroxycarbonyl,
C₁-C₆ alkyl, C₁-C₆ alkenyl, C₁-C₆ alkynyl,
C₁-C₆ alkoxy, hydroxyC₁-C₆ alkyl, C₁-C₆ alkoxyC₁-C₆ alkyl, perfluoro C₁-C₆ alkyl, perfluoroC₁-C₆ alkoxy,
C₁-C₆ alkylamino, di-C₁-C₆ alkylamino, aminoC₁-C₆ alkyl, C₁-C₆ alkylaminoC₁-C₆ alkyl, di-C₁-C₆ alkylaminoC₁-C₆ alkyl,
C₁-C₆acyl, C₁-C₆acyloxy, C₁-C₆acyloxyC₁-C₆ alkyl, C₁-C₆ acylamino,
C₁-C₆ alkylthio, C₁-C₆ alkylthiocarbonyl, C₁-C₆ alkylthioxo, C₁-C₆ alkoxycarbonyl,
C₁-C₆ alkylsulfonyl, C₁-C₆ alkylsulfonylamino,
aminosulfonyl, C₁-C₆ alkylaminosulfonyl, di-C₁-C₆ alkylaminosulfonyl,
3-8 membered cycloalkyl, 4-8 membered heterocycloalkyl, phenyl and monocyclic heteroaryl;
   or a pharmaceutically acceptable salt thereof.

Conventional antipsychotics are antagonists of dopamine (D₂) receptors. The atypical antipsychotics also have D₂ antagonistic properties but possess different binding kinetics to these receptors and activity at other receptors, particularly 5-HT_{2A}, 5-HT_{2C} and 5-HT_{2D} (Schmidt B et al, Soc. Neurosci. Abstr. 24:2177, 1998).

The class of atypical antipsychotics includes clozapine (clozaril®D), 8-chloro-11-(4-methyl-1-piperazinyl)-5H-dibenzo[b,e][1,4]diazepine (U.S. Pat. No. 3,539,573); risperidone (risperdal®), 3-[2-[4-(6-fluoro-1,2-benzisoxazol-3-yl)piperidino]ethyl]-2-methyl-6,7,8,9-tetrahydro-4H-pyrido-[1,2-a]pyrimidin-4-one (U.S. Pat. No. 4,804,663); olanzapine (zyprexa®), 2-methyl-4-(4-methyl-1-piperazinyl)-10H-thieno[2,3-b][1,5]benzodiazepine (U.S. Pat. No. 5,229,382); quetiapine (seroquel®), 5-[2-(4-dibenzo[b,f][1,4]thiazepin-11-yl-1-piperazinyl)ethoxy]ethanol (U.S. Pat. No. 4,879,288); aripiprazole (abilify®), 7-{4-[4-(2,3-dichlorophenyl)-1-piperazinyl]-butoxy}-3,4-dihydro carbostyril and 7-{4-[4-(2,3-dichlorophenyl)-1-piperazinyl]-butoxy}-3,4-dihydro-2(1H)-quinolinone (U.S. Pat. Nos. 4,734,416 and 5,006,528); sertindole, 1-[2-[4-[5-chloro-1-(4-fluorophenyl)-1H-indol-3-yl]-1-piperidinyl]ethyl]imidazolidin-2-one (U.S. Pat. No. 4,710,500); amisulpride (U.S. Pat. No. 4,410,822); ziprasidone (geodon®), 5-[2-[4-(1,2-benzisothiazol-3-yl)piperazin-3-yl]ethyl]-6-chloroindolin-2-one hydrochloride hydrate (U.S. Pat. No. 4,831,031); asenapine, trans-5-Chloro-2,3,3a,12b-tetrahydro-2-methyl-1H-dibenz [2,3:6,7]oxepino [4,5-c]pyrrole maleate; (3R,4R,5R)-3-amino-4,5-dimethyl-heptanoic acid and (3R,4R,5R)-3-amino-4,5-dimethyl-octanoic acid (PCT Application No. PCT/IB2004/002985, not published at the date of filing).

The contents of all patents and publications cited within the present application are hereby incorporated by reference.

### SUMMARY OF THE INVENTION

It has now been found that combination therapy with an alpha-2-delta ligand and an atypical antipsychotic results in improvement in the treatment of pain. Furthermore, when administered simultaneously, sequentially or separately, the alpha-2-delta ligand and atypical antipsychotic may interact in a synergistic manner to control pain. This synergy allows a reduction in the dose required of each compound, leading to a reduction in the side effects and enhancement of the clinical utility of the compounds.

Accordingly, the invention provides, as a first aspect, a combination product comprising an alpha-2-delta ligand and an atypical antipsychotic.

As an alternative or further aspect, the invention provides a synergistic combination product comprising an alpha-2-delta ligand and an atypical antipsychotic.

Useful cyclic alpha-2-delta ligands of the present invention are illustrated by the following formula (I): 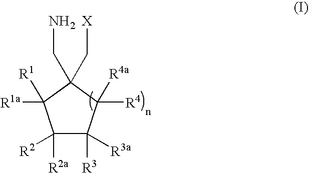
wherein X is a carboxylic acid or carboxylic acid bioisostere;
n is 0, 1 or 2; and
R¹, R^{1a}, R², R^{2a}, R³, R^{3a}, R⁴ and R^{4a} are independently selected from H and C₁-C₆ alkyl, or
R¹ and R² or R² and R³ are taken together to form a C₃-C₇ cycloalkyl ring, which is optionally substituted with one or two substituents selected from C₁-C₆ alkyl, or a pharmaceutically acceptable salt thereof.

In formula (I), suitably, R¹, R^{1a}, R^{2a}, R^{3a}, R⁴ and R^{4a} are H and R² and R³ are independently selected from H and methyl, or R^{1a}, R^{2a}, R^{3a} and R^{4a} are H and R¹ and R² or R² and R³ are taken together to form a C₃-C₇ cycloalkyl ring, which is optionally substituted with one or two methyl substituents. A suitable carboxylic acid bioisostere is selected from tetrazolyl and oxadiazolonyl. X is preferably a carboxylic acid.

In formula (I), preferably, R¹, R^{1a}, R^{2a}, R^{3a}, R⁴ and R^{4a} are H and R² and R³ are independently selected from H and methyl, or R^{1a}, R^{2a}, R^{3a} and R^{4a} are H and R¹ and R² or R² and R³ are taken together to form a C₄-C₅ cycloalkyl ring, or, when n is 0, R¹, R^{1a}, R^{2a}, R^{3a}, R⁴ and R^{4a} are H and R² and R³ form a cyclopentyl ring, or, when n is 1, R¹, R^{1a}, R^{2a}, R^{3a}, R⁴ and R^{4a} are H and R² and R³ are both methyl or R¹, R^{1a}, R^{2a}, R^{3a}, R⁴ and R^{4a} are H and R² and R³ form a cyclobutyl ring, or, when n is 2, R¹, R^{1a}, R², R^{2a}, R³, R^{3a}, R⁴ and R^{4a} are H, or, n is 0, R¹, R^{1a}, R^{2a}, R^{3a}, R⁴ and R^{4a} are H and R² and R³ form a cyclopentyl ring.

Useful acyclic alpha-2-delta ligands of the present invention are illustrated by the following formula (II):

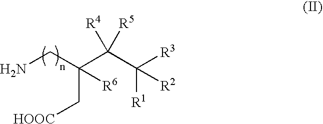

wherein:

n is 0 or 1, R¹ is hydrogen or (C₁-C₆)alkyl; R² is hydrogen or (C₁-C₆)alkyl; R³ is hydrogen or (C₁-C₆)alkyl; R⁴ is hydrogen or (C₁-C₆)alkyl; R⁵ is hydrogen or (C₁-C₆)alkyl and R² is hydrogen or (C₁-C₆)alkyl, or a pharmaceutically acceptable salt thereof.

According to formula (II), suitably R¹ is C₁-C₆ alkyl, R² is methyl, R³-R⁶ are hydrogen and n is 0 or 1. More suitably R¹ is methyl, ethyl, n-propyl or n-butyl, R² is methyl, R³-R⁶ are hydrogen and n is 0 or 1. When R² is methyl, R³-R⁶ are hydrogen and n is 0, R¹ is suitably ethyl, n-propyl or n-butyl. When R² is methyl, R³-R⁶ are hydrogen and n is 1, R¹ is suitably methyl or n-propyl. Compounds of formula (II) are suitably in the 3S,5R configuration.

Examples of alpha-2-delta ligands for use with the present invention are those compounds generally or specifically disclosed in U.S. Pat. No. 4,024,175, particularly gabapentin, EP641330, particularly pregabalin, U.S. Pat. No. 5,563,175, WO9733858, WO9733859, WO9931057, WO9931074, WO9729101, WO02085839, particularly [(1R,5R,6S)-6-(Aminomethyl)bicyclo[3.2.0]hept-6-yl]acetic acid, WO9931075, particularly 3-(1-Aminomethyl-cyclohexylmethyl)-4H-[1,2,4]oxadiazol-5-one and C-[1-(1H-Tetrazol-5-ylmethyl)-cycloheptyl]-methylamine, WO9921824, particularly (3S,4S)-(1-Aminomethyl-3,4-dimethyl-cyclopentyl)-acetic acid, WO0190052, WO0128978, particularly (1α,3α,5α)(3-amino-methyl-bicyclo[3.2.0]hept-3-yl)-acetic acid, EP0641330, WO9817627, WO0076958, particularly (3S,5R)-3-aminomethyl-5-methyl-octanoic acid, PCT/IB03/00976, particularly (3S,5R)-3-amino-5-methyl-heptanoic acid, (3S,5R)-3-amino-5-methyl-nonanoic acid and (3S,5R)-3-Amino-5-methyl-octanoic acid, WO2004/039367, particularly (2S,4S)-4-(3-fluoro-phenoxymethyl)-pyrrolidine-2-carboxylic acid, (2S,4S)-4-(2,3-difluoro-benzyl)-pyrrolidine-2-carboxylic acid, (2S,4S)-4-(3-chlorophenoxy)proline and (2S,4S)-4-(3-fluorobenzyl)proline, EP1178034, EP1201240, WO9931074, WO03000642, WO0222568, WO0230871, WO0230881, WO02100392, WO02100347, WO0242414, WO0232736 and WO0228881 or pharmaceutically acceptable salts thereof, all of which are incorporated herein by reference.

Preferred alpha-2-delta ligands of the present invention include: gabapentin, pregabalin, [(1R,5R,6S)-6-(Aminomethyl)bicyclo[3.2.0]hept-6-yl]acetic acid, 3-(1-Aminomethyl-cyclohexylmethyl)-4H-[1,2,4]oxadiazol-5-one, (3S,4S)-(1-Aminomethyl-3,4-dimethyl-cyclopentyl)-acetic acid, (1α,3α,5α)(3-amino-methyl-bicyclo[3.2.0]hept-3-yl)-acetic acid, (3S,5R)-3-Aminomethyl-5-methyl-octanoic acid, (3S,5R)-3-amino-5-methyl-heptanoic acid, (3S,5R)-3-amino-5-methyl-nonanoic acid, (3S,5R)-3-Amino-5-methyl-octanoic acid, (2S,4S)-4-(3-fluoro-phenoxymethyl)-pyrrolidine-2-carboxylic acid, (2S,4S)-4-(2,3-difluoro-benzyl)-pyrrolidine-2-carboxylic acid, (2S,4S)-4-(3-chlorophenoxy)proline and (2S,4S)-4-(3-fluorobenzyl)proline, or pharmaceutically acceptable salts thereof. Particularly preferred alpha-2-delta ligands of the present invention are selected from gabapentin, pregabalin, (1α,3α,5α)(3-amino-methyl-bicyclo[3.2.0]hept-3-yl)-acetic acid, ((2S,4S)-4-(3-chlorophenoxy)proline and (2S,4S)-4-(3-fluorobenzyl)proline, or pharmaceutically acceptable salts thereof.

Atypical antipsychotics useful according to the present invention include those comprised within the disclosure of U.S. Pat. No. 4,831,031, i.e. the compounds of formula (I): 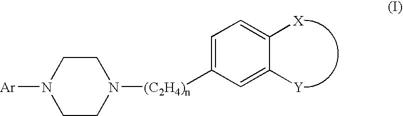
wherein Ar is naphthyl optionally substituted by fluoro, chloro, trifluoromethyl, methoxy, cyano or nitro; quinolyl; isoquinolyl; 6-hydroxy-8-quinolyl; benzoisothiazolyl or an oxide or dioxide thereof each optioannly substituted by fluoro, chloro, trifluoromethyl, methoxy, cyano or nitro; benzothiazolyl; benzothiadiazolyl; benzotriazolyl; benzoxazolyl; benzoxazolonyl; indolyl; indanyl optionally substituted by one or two fluoro; 3-indazolyl optionally substituted by 1-trifluoromethylphenyl; or phthalazinyl;
n is 1 or 2; and
X and Y together with the phenyl to which they are attached form quinolyl; 2-hydroxyquinolyl; benzothiazolyl; 2-aminobenzothiazolyl; benzoisothiazolyl; indazolyl; 3-hydroxyindazolyl; indolyl; spiro[cyclopentane-1,3′-indolinyl]; oxindolyl optionally substituted by one to three of (C₁-C₃)alkyl, or one of chloro, fluoro or phenyl, said phenyl being optionally substituted by one chloro or fluoro; benzoxazolyl; 2-aminobenzoxazolyl; benzoxazolonyl; 2-aminobenzoxazolinyl; benzothiazolonyl; benzoimidazolonyl; or benzotriazolyl.
   A particular preferred compound of formula (I) is ziprasidone.

Examples of atypical antipsychotics for use in the present invention are the compounds generically and specifically disclosed in U.S. Pat. No. 4,831,301, particularly ziprasidone; U.S. Pat. No. 5,229,382, particularly olanzapine; U.S. Pat. No. 3,539,573, particularly clozapine; U.S. Pat. No. 4,804,663, particularly risperidone; U.S. Pat. No. 4,710,500, particularly sertindole; U.S. Pat. No. 4,879,288, particularly quetiapine; U.S. Pat. No. 4,734,416, particularly aripiprazole; U.S. Pat. No. 4,401,822, particularly amisulpride; PCT Application No. PCT/IB2004/002985, particularly (3R,4R,5R)-3-amino-4,5-dimethyl-heptanoic acid and (3R,4R,5R)-3-amino-4,5-dimethyl-octanoic acid; and asenapine; or pharmaceutically acceptable salts thereof, all of which are incorporated herein by reference.

Suitable atypical antipsychotics for use in the present invention include ziprasidone, olanzapine, clozapine, risperidone, sertindole, quetiapine, aripiprazole, asenapine, amisulpride, (3R,4R,5R)-3-amino-4,5-dimethyl-heptanoic acid and (3R,4R,5R)-3-amino-4,5-dimethyl-octanoic acid, or pharmaceutically acceptable salts thereof. Preferably the atypical antipsychotic is ziprasidone, or a pharmaceutically acceptable salt thereof.

The suitability of any particular atypical antipsychotic can be readily determined by evaluation of its potency and selectivity using literature methods followed by evaluation of its toxicity, absorption, metabolism, pharmacokinetics, etc in accordance with standard pharmaceutical practices.

As an alternative or further aspect of the present invention, there is provided a combination comprising gabapentin, or a pharmaceutically acceptable salt thereof, and an atypical antipsychotic selected from ziprasidone, olanzapine, clozapine, risperidone, sertindole, quetiapine, aripiprazole, asenapine, amisulpride, (3R,4R,5R)-3-amino-4,5-dimethyl-heptanoic acid and (3R,4R,5R)-3-amino-4,5-dimethyl-octanoic acid, or a pharmaceutically acceptable salt thereof. A particularly preferred combination comprises gabapentin and ziprasidone, and their pharmaceutically acceptable salts.

As an alternative or further aspect of the present invention, there is provided a combination comprising pregabalin and an atypical antipsychotic selected from ziprasidone, olanzapine, clozapine, risperidone, sertindole, quetiapine, aripiprazole, asenapine, amisulpride, (3R,4R,5R)-3-amino-4,5-dimethyl-heptanoic acid and (3R,4R,5R)-3-amino-4,5-dimethyl-octanoic acid, and their pharmaceutically acceptable salts. A particularly preferred combination comprises pregabalin and ziprasidone, and their pharmaceutically acceptable salts.

As an alternative or further aspect of the present invention, there is provided a combination comprising (1α,3α,5α)(3-amino-methyl-bicyclo[3.2.0]hept-3-yl)-acetic acid or a pharmaceutically acceptable salt thereof, and an atypical antipsychotic. Suitably, there is provided a combination comprising (1α,3α,5α)(3-amino-methyl-bicyclo[3.2.0]hept-3-yl)-acetic acid or a pharmaceutically acceptable salt thereof, and an atypical antipsychotic selected from ziprasidone, olanzapine, clozapine, risperidone, sertindole, quetiapine, aripiprazole, asenapine, amisulpride, (3R,4R,5R)-3-amino-4,5-dimethyl-heptanoic acid and (3R,4R,5R)-3-amino-4,5-dimethyl-octanoic acid, or a pharmaceutically acceptable salt thereof. A particularly preferred combination comprises (1α,3α,5α)(3-amino-methyl-bicyclo[3.2.0]hept-3-yl)-acetic acid and ziprasidone, and their pharmaceutically acceptable salts.

As an alternative or further aspect of the present invention, there is provided a combination comprising (2S,4S)-4-(3-chlorophenoxy)proline or a pharmaceutically acceptable salt thereof, and an atypical antipsychotic. Suitably, there is provided a combination comprising (2S,4S)-4-(3-chlorophenoxy)proline or a pharmaceutically acceptable salt thereof, and an atypical antipsychotic selected from ziprasidone, olanzapine, clozapine, risperidone, sertindole, quetiapine, aripiprazole, asenapine, amisulpride, (3R,4R,5R)-3-amino-4,5-dimethyl-heptanoic acid and (3R,4R,5R)-3-amino-4,5-dimethyl-octanoic acid, or a pharmaceutically acceptable salt thereof. A particularly preferred combination comprises (2S,4S)-4-(3-chlorophenoxy)proline and ziprasidone, and their pharmaceutically acceptable salts.

As an alternative or further aspect of the present invention, there is provided a combination comprising (2S,4S)-4-(3-fluorobenzyl)proline or a pharmaceutically acceptable salt thereof, and an atypical antipsychotic. Suitably, there is provided a combination comprising (2S,4S)-4-(3-fluorobenzyl)proline or a pharmaceutically acceptable salt thereof, and an atypical antipsychotic selected from ziprasidone, olanzapine, clozapine, risperidone, sertindole, quetiapine, aripiprazole, asenapine, amisulpride, (3R,4R,5R)-3-amino-4,5-dimethyl-heptanoic acid and (3R,4R,5R)-3-amino-4,5-dimethyl-octanoic acid, or a pharmaceutically acceptable salt thereof. A particularly preferred combination comprises (2S,4S)-4-(3-fluorobenzyl)proline and ziprasidone, and their pharmaceutically acceptable salts.

As a yet further preferred aspect of the present invention, the combination is selected from:
gabapentin and ziprasidone;
gabapentin and olanzapine;
gabapentin and clozapine;
gabapentin and risperidone;
gabapentin and sertindole;
gabapentin and quetiapine;
gabapentin and aripiprazole;
gabapentin and asenapine;
gabapentin and amisulpride;
gabapentin and (3R,4R,5R)-3-amino-4,5-dimethyl-heptanoic acid;
gabapentin and (3R,4R,5R)-3-amino-4,5-dimethyl-octanoic acid;
pregabalin and ziprasidone;
pregabalin and olanzapine;
pregabalin and clozapine;
pregabalin and risperidone;
pregabalin and sertindole;
pregabalin and quetiapine;
pregabalin and aripiprazole;
pregabalin and asenapine;
pregabalin and amisulpride;
pregablin and (3R,4R,5R)-3-amino-4,5-dimethyl-heptanoic acid;
pregabalin and (3R,4R,5R)-3-amino-4,5-dimethyl-octanoic acid
[(1R,5R,6S)-6-(Aminomethyl)bicyclo[3.2.0]hept-6-yl]acetic acid and ziprasidone;
[(1R,5R,6S)-6-(Aminomethyl)bicyclo[3.2.0]hept-6-yl]acetic acid and olanzapine;
[(1R,5R,6S)-6-(Aminomethyl)bicyclo [3.2.0]hept-6-yl]acetic acid and clozapine;
[(1R,5R,6S)-6-(Aminomethyl)bicyclo[3.2.0]hept-6-yl]acetic acid and risperidone;
[(1R,5R,6S)-6-(Aminomethyl)bicyclo[3.2.0]hept-6-yl]acetic acid and sertindole;
[(1R,5R,6S)-6-(Aminomethyl)bicyclo[3.2.0]hept-6-yl]acetic acid and quetiapine;
[(1R,5R,6S)-6-(Aminomethyl)bicyclo[3.2.0]hept-6-yl]acetic acid and aripiprazole;
[(1R,5R,6S)-6-(Aminomethyl)bicyclo[3.2.0]hept-6-yl]acetic acid and asenapine;
[(1R,5R,6S)-6-(Aminomethyl)bicyclo[3.2.0]hept-6-yl]acetic acid and amisulpride;
[(1R,5R,6S)-6-(Aminomethyl)bicyclo[3.2.0]hept-6-yl]acetic acid and (3R,4R,5R)-3-amino-4,5-dimethyl-heptanoic acid;
[(1R,5R,6S)-6-(Aminomethyl)bicyclo[3.2.0]hept-6-yl]acetic acid and (3R,4R,5R)-3-amino-4,5-dimethyl-octanoic acid;
(1α,3α,5α)(3-amino-methyl-bicyclo[3.2.0]hept-3-yl)-acetic acid and ziprasidone;
(1α,3α,5α)(3-amino-methyl-bicyclo[3.2.0]hept-3-yl)-acetic acid and olanzapine;
(1α,3α,5α)(3-amino-methyl-bicyclo[3.2.0]hept-3-yl)-acetic acid and clozapine;
(1α,3α,5α)(3-amino-methyl-bicyclo[3.2.0]hept-3-yl)-acetic acid and risperidone;
(1α,3α,5α)(3-amino-methyl-bicyclo[3.2.0]hept-3-yl)-acetic acid and sertindole;
(1α,3α,5α)(3-amino-methyl-bicyclo[3.2.0]hept-3-yl)-acetic acid and quetiapine;
(1α,3α,5α)(3-amino-methyl-bicyclo[3.2.0]hept-3-yl)-acetic acid and aripiprazole;
(1α,3α,5α)(3-amino-methyl-bicyclo[3.2.0]hept-3-yl)-acetic acid and asenapine;
(1α,3α,5α)(3-amino-methyl-bicyclo[3.2.0]hept-3-yl)-acetic acid and amisulpride;
(1α,3α,5α)(3-amino-methyl-bicyclo[3.2.0]hept-3-yl)-acetic acid and (3R,4R,5R)-3-amino-4,5-dimethyl-heptanoic acid;
(1α,3α,5α)(3-amino-methyl-bicyclo[3.2.0]hept-3-yl)-acetic acid and (3R,4R,5R)-3-amino-4,5-dimethyl-octanoic acid;
(3S,4S)-(1-Aminomethyl-3,4-dimethyl-cyclopentyl)-acetic acid and ziprasidone;
(3S,4S)-(1-Aminomethyl-3,4-dimethyl-cyclopentyl)-acetic acid and olanzapine;
(3S,4S)-(1-Aminomethyl-3,4-dimethyl-cyclopentyl)-acetic acid and clozapine;
(3S,4S)-(1-Aminomethyl-3,4-dimethyl-cyclopentyl)-acetic acid and risperidone;
(3S,4S)-(1-Aminomethyl-3,4-dimethyl-cyclopentyl)-acetic acid and sertindole;
(3S,4S)-(1-Aminomethyl-3,4-dimethyl-cyclopentyl)-acetic acid and quetiapine;
(3S,4S)-(1-Aminomethyl-3,4-dimethyl-cyclopentyl)-acetic acid and aripiprazole;
(3S,4S)-(1-Aminomethyl-3,4-dimethyl-cyclopentyl)-acetic acid and asenapine;
(3S,4S)-(1-Aminomethyl-3,4-dimethyl-cyclopentyl)-acetic acid and amisulpride;
(3S,4S)-(1-Aminomethyl-3,4-dimethyl-cyclopentyl)-acetic acid and (3R,4R,5R)-3-amino-4,5-dimethyl-heptanoic acid;
(3S,4S)-(1-Aminomethyl-3,4-dimethyl-cyclopentyl)-acetic acid and (3R,4R,5R)-3-amino-4,5-dimethyl-octanoic acid;
(2S,4S)-4-(3-chlorophenoxy)proline and ziprasidone;
(2S,4S)-4-(3-chlorophenoxy)proline and olanzapine;
(2S,4S)-4-(3-chlorophenoxy)proline and clozapine;
(2S,4S)-4-(3-chlorophenoxy)proline and risperidone;
(2S,4S)-4-(3-chlorophenoxy)proline and sertindole;
(2S,4S)-4-(3-chlorophenoxy)proline and quetiapine;
(2S,4S)-4-(3-chlorophenoxy)proline and aripiprazole;
(2S,4S)-4-(3-chlorophenoxy)proline and asenapine;
(2S,4S)-4-(3-chlorophenoxy)proline and amisulpride;
(2S,4S)-4-(3-chlorophenoxy)proline and (3R,4R,5R)-3-amino-4,5-dimethyl-heptanoic acid;
(2S,4S)-4-(3-chlorophenoxy)proline and (3R,4R,5R)-3-amino-4,5-dimethyl-octanoic acid;
(2S,4S)-4-(3-fluorobenzyl)proline and ziprasidone;
(2S,4S)-4-(3-fluorobenzyl)proline and olanzapine;
(2S,4S)-4-(3-fluorobenzyl)proline and clozapine;
(2S,4S)-4-(3-fluorobenzyl)proline and risperidone;
(2S,4S)-4-(3-fluorobenzyl)proline and sertindole;
(2S,4S)-4-(3-fluorobenzyl)proline and quetiapine;
(2S,4S)-4-(3-fluorobenzyl)proline and aripiprazole;
(2S,4S)-4-(3-fluorobenzyl)proline and asenapine;
(2S,4S)-4-(3-fluorobenzyl)proline and amisulpride;
(2S,4S)-4-(3-fluorobenzyl)proline and (3R,4R,5R)-3-amino-4,5-dimethyl-heptanoic acid; and
(2S,4S)-4-(3-fluorobenzyl)proline and (3R,4R,5R)-3-amino-4,5-dimethyl-octanoic acid;
   or pharmaceutically acceptable salts or solvates of either or both components of any such combination.

Particularly preferred combinations of the invention include those in which each variable of the combination is selected from the suitable parameters for each variable. Even more preferable combinations of the invention include those where each variable of the combination is selected from the more suitable, most suitable, preferred or more preferred parameters for each variable.

The combination of the present invention in a single dosage form is suitable for administration to any mammalian subject, preferably human. Administration may be once (o.d.), twice (b.i.d.) or three times (t.i.d.) daily, suitably b.i.d. or t.i.d., more suitably b.i.d, most suitably o.d.

Thus, as a further aspect of the present invention, there is provided the use of a combination, particularly synergistic, of an alpha-2-delta ligand and an atypical antipsychotic in the manufacture of a once, twice or thrice, suitably twice or thrice, more suitably twice, most suitably once daily administration medicament for the curative, prophylactic or palliative treatment of pain.

Determining a synergistic interaction between one or more components, the optimum range for the effect and absolute dose ranges of each component for the effect may be definitively measured by administration of the components over different w/w ratio ranges and doses to patients in need of treatment. For humans, the complexity and cost of carrying out clinical studies on patients renders impractical the use of this form of testing as a primary model for synergy. However, the observation of synergy in one species can be predictive of the effect in other species and animal models exist, as described herein, to measure a synergistic effect and the results of such studies can also be used to predict effective dose and plasma concentration ratio ranges and the absolute doses and plasma concentrations required in other species by the application of pharmacokinetic/pharmacodynamic methods. Established correlations between animal models and effects seen in man suggest that synergy in animals is best-demonstrated using static and dynamic allodynia measurements in rodents that have undergone surgical (e.g. chronic constriction injury) or chemical (e.g. streptozocin) procedures to induce the allodynia. Because of plateau effects in such models, their value is best assessed in terms of synergistic actions that in neuropathic pain patients would translate to dose-sparing advantages. Other models in which existing agents used for the treatment of neuropathic pain give only a partial response are more suited to predict the potential of combinations acting synergistically to produce increased maximal efficacy at maximally tolerated doses of the two components.

Thus, as a further aspect of the present invention, there is provided a synergistic combination for human administration comprising an alpha-2-delta ligand and an atypical antipsychotic, or pharmaceutically acceptable salts or solvates thereof, in a w/w combination range which corresponds to the absolute ranges observed in a non-human animal model, preferably a rat model, primarily used to identify a synergistic interaction. Suitably, the ratio range in humans corresponds to a non-human range selected from between 1:50 to 50:1 parts by weight, 1:50 to 20:1, 1:50 to 10:1, 1:50 to 1:1, 1:20 to 50:1, 1:20 to 20:1, 1:20 to 10:1, 1:20 to 1:1, 1:10 to 50:1, 1:10 to 20:1, 1:10 to 10:1, 1:10 to 1:1, 1:1 to 50:1, 1.1 to 20:1 and 1:1 to 10:1. More suitably, the human range corresponds to a non-human range of 1:10 to 20:1 parts by weight. Preferably, the human range corresponds to a synergistic non-human range of the order of 1:1 to 10:1 parts by weight.

For humans, several experimental pain models may be used in man to demonstrate that agents with proven synergy in animals also have effects in man compatible with that synergy. Examples of human models that may be fit for this purpose include the heat/capsaicin model (Petersen, K. L. & Rowbotham, M. C. (1999) NeuroReport 10, 1511-1516), the i.d capsaicin model (Andersen, O. L., Felsby, S., Nicolaisen, L., Bjerring, P., Jsesn, T. S. & Arendt-Nielsen, L. (1996) Pain 66, 51-62), including the use of repeated capsaicin trauma (Witting, N., Svesson, P., Arendt-Nielsen, L. & Jensen, T. S. (2000) Somatosensory Motor Res. 17, 5-12), and summation or wind-up responses (Curatolo, M. et al. (2000) Anesthesiology 93, 1517-1530). With these models, subjective assessment of pain intensity or areas of hyperalgesia may be used as endpoints, or more objective endpoints, reliant on electrophysiological or imaging technologies (such as functional magnetic resonance imaging) may be employed (Bornhovd, K., Quante, M., Glauche, V., Bromm, B., Weiller, C. & Buchel, C. (2002) Brain 125, 1326-1336). All such models require evidence of objective validation before it can be concluded that they provide evidence in man of supporting the synergistic actions of a combination that have been observed in animal studies.

For the present invention in humans, a suitable alpha-2-delta ligand:atypical antipsychotic ratio range is selected from between 1:50 to 50:1 parts by weight, 1:50 to 20:1, 1:50 to 10:1, 1:50 to 1:1, 1:20 to 50:1, 1:20 to 20:1, 1:20 to 10:1, 1:20 to 1:1, 1:10 to 50:1, 1:10 to 20:1, 1:10 to 10:1, 1:10 to 1:1, 1:1 to 50:1, 1.1 to 20:1 and 1:1 to 10:1, more suitably 1:10 to 20:1, preferably, 1:1 to 10:1.

Optimal doses of each component for synergy can be determined according to published procedures in animal models. However, in man (even in experimental models of pain) the cost can be very high for studies to determine the entire exposure-response relationship at all therapeutically relevant doses of each component of a combination. It may be necessary, at least initially, to estimate whether effects can be observed that are consistent with synergy at doses that have been extrapolated from those that give optimal synergy in animals. In scaling the doses from animals to man, factors such as relative body weight/body surface area, relative absorption, distribution, metabolism and excretion of each component and relative plasma protein binding need to be considered and, for these reasons, the optimal dose ratio predicted for man (and also for patients) is unlikely to be the same as the dose ratio shown to be optimal in animals. However, the relationship between the two can be understood and calculated by one skilled in the art of animal and human pharmacokinetics. Important in establishing the bridge between animal and human effects are the plasma concentrations obtained for each component used in the animal studies, as these are related to the plasma concentration of each component that would be expected to provide efficacy in man. Pharmacokinetic/pharmacodynamic modeling (including methods such as isobolograms, interaction index and response surface modelling) and simulations may help to predict synergistic dose ratios in man, particularly where either or both of these components has already been studied in man.

It is important to ascertain whether any concluded synergy observed in animals or man is due solely to pharmacokinetic interactions. For example, inhibition of the metabolism of one compound by another might give a false impression of pharmacodynamic synergy

Thus, according to a further aspect of the present invention, there is provided a synergistic combination for administration to humans comprising an alpha-2-delta ligand and an atypical antipsychotic or pharmaceutically acceptable salts or solvates thereof, where the dose range of each component corresponds to the absolute ranges observed in a non-human animal model, preferably the rat model, primarily used to identify a synergistic interaction.

Suitably, the dose of alpha-2-delta ligand for use in a human is in a range selected from 1-1200 mg, 1-500 mg, 1-100 mg, 1-50 mg, 1-25 mg, 500-1200 mg, 100-1200 mg, 100-500 mg, 50-1200 mg, 50-500 mg, or 50-100 mg, suitably 50-100 mg, b.i.d. or t.i.d., suitably t.i.d., and the dose of atypical antipsychotic is in a range selected from 1-200 mg, 1-100 mg, 0.25-25 mg, 1-50 mg, 1-25 mg, 10-100 mg, 10-50 mg or 10-25 mg, suitably 10-100 mg, b.i.d or t.i.d, suitably t.i.d.

It will be apparent to the skilled reader that the plasma concentration ranges of the alpha-2-delta ligand and atypical antipsychotic combinations of the present invention required to provide a therapeutic effect depend on the species to be treated, and components used. For example, for gabapentin in the rat, the Cmax values range from 0.520 μg/ml to 10.5 μg/ml.

It is possible, using standard PK/PD and allometric methods, to extrapolate the plasma concentration values observed in an animal model to predict the values in a different species, particularly human. Thus, as a further aspect of the present invention, there is provided a synergistic combination for administration to humans comprising an alpha-2-delta ligand and an atypical antipsychotic, where the plasma concentration range of each component corresponds to the absolute ranges observed in a non-human animal model, preferably the rat model, primarily used to identify a synergistic interaction. Suitably, the plasma concentration range in the human corresponds to a range of 0.05 μg/ml to 10.5 μg/ml for an alpha-2-delta ligand in the rat model.

Particularly preferred combinations of the invention include those in which each variable of the combination is selected from the suitable parameters for each variable. Even more preferable combinations of the invention include those where each variable of the combination is selected from the more suitable, most suitable, preferred or more preferred parameters for each variable.

### DETAILED DESCRIPTION OF THE INVENTION

The compounds of the present invention are prepared by methods well known to those skilled in the art. Specifically, the patents, patent applications and publications, mentioned hereinabove, each of which is hereby incorporated herein by reference, exemplify compounds which can be used in the combinations, pharmaceutical compositions, methods and kits in accord with the present invention, and refer to methods of preparing those compounds.

The compounds of the present combination invention can exist in unsolvated forms as well as solvated forms, including hydrated forms. In general, the solvated forms, including hydrated forms, which may contain isotopic substitutions (e.g. D20, d6-acetone, d6-DMSO), are equivalent to unsolvated forms and are encompassed within the scope of the present invention.

Certain of the compounds of the present invention possess one or more chiral centers and each center may exist in the R or S configuration. The present invention includes all enantiomeric and epimeric forms as well as the appropriate mixtures thereof. Separation of diastereoisomers or cis and trans isomers may be achieved by conventional techniques, e.g. by fractional crystallisation, chromatography or H.P.L.C. of a stereoisomeric mixture of a compound of the invention or a suitable salt or derivative thereof.

A number of the alpha-2-delta ligands of the present invention are amino acids. Since amino acids are amphoteric, pharmacologically compatible salts can be salts of appropriate non-toxic inorganic or organic acids or bases. Suitable acid addition salts are the acetate, aspartate, benzoate, besylate, bicarbonate/carbonate, bisulphate, camsylate, citrate, edisylate, esylate, fumarate, gluceptate, gluconate, glucuronate, hibenzate, hydrochloride/chloride, hydrobromide/bromide, hydroiodide/iodide, hydrogen phosphate, isethionate, D- and L-lactate, malate, maleate, malonate, mesylate, methylsulphate, 2-napsylate, nicotinate, nitrate, orotate, palmoate, phosphate, saccharate, stearate, succinate sulphate, D- and L-tartrate, and tosylate salts. Suitable base salts are formed from bases which form non-toxic salts and examples are the sodium, potassium, aluminium, calcium, magnesium, zinc, choline, diolamine, olamine, arginine, glycine, tromethamine, benzathine, lysine, meglumine and diethylamine salts. Salts with quaternary ammonium ions can also be prepared with, for example, the tetramethyl-ammonium ion. The compounds of the invention may also be formed as a zwitterion.

A suitable salt for amino acid compounds of the present invention is the hydrochloride salt. For a review on suitable salts see Stahl and Wermuth, Handbook of Pharmaceutical Salts: Properties, Selection, and Use, Wiley-VCH, Weinheim, Germany (2002).

Also within the scope of the invention are clathrates, drug-host inclusion complexes wherein, in contrast to the aforementioned solvates, the drug and host are present in non-stoichiometric amounts. For a review of such complexes, see J Pharm Sci, 64 (8), 1269-1288 by Haleblian (August 1975).

Hereinafter all references to compounds of the invention include references to salts thereof and to solvates and clathrates of compounds of the invention and salts thereof.

Also included within the present scope of the compounds of the invention are polymorphs thereof.

Prodrugs of the above compounds of the invention are included in the scope of the instant invention. The chemically modified drug, or prodrug, should have a different pharmacokinetic profile to the parent, enabling easier absorption across the mucosal epithelium, better salt formulation and/or solubility, improved systemic stability (for an increase in plasma half-life, for example). These chemical modifications may be
(1) Ester or amide derivatives which may be cleaved by, for example, esterases or lipases. For ester derivatives, the ester is derived from the carboxylic acid moiety of the drug molecule by known means. For amide derivatives, the amide may be derived from the carboxylic acid moiety or the amine moiety of the drug molecule by known means.
(2) Peptides which may be recognized by specific or nonspecific proteinases. A peptide may be coupled to the drug molecule via amide bond formation with the amine or carboxylic acid moiety of the drug molecule by known means.
(3) Derivatives that accumulate at a site of action through membrane selection of a prodrug form or modified prodrug form.
(4) Any combination of 1 to 3.

Aminoacyl-glycolic and -lactic esters are known as prodrugs of amino acids (Wermuth C. G., Chemistry and Industry, 1980:433-435). The carbonyl group of the amino acids can be esterified by known means. Prodrugs and soft drugs are known in the art (Palomino E., Drugs of the Future, 1990; 15(4):361-368). The last two citations are hereby incorporated by reference.

The combination of the present invention is useful for the general treatment of pain, particularly neuropathic pain. Physiological pain is an important protective mechanism designed to warn of danger from potentially injurious stimuli from the external environment. The system operates through a specific set of primary sensory neurones and is exclusively activated by noxious stimuli via peripheral transducing mechanisms (Millan 1999 Prog. Neurobio. 57: 1-164 for an integrative Review). These sensory fibres are known as nociceptors and are characterised by small diameter axons with slow conduction velocities. Nociceptors encode the intensity, duration and quality of noxious stimulus and by virtue of their topographically organised projection to the spinal cord, the location of the stimulus. The nociceptors are found on nociceptive nerve fibres of which there are two main types, A-delta fibres (myelinated) and C fibres (non-myelinated). The activity generated by nociceptor input is transferred after complex processing in the dorsal horn, either directly or via brain stem relay nuclei to the ventrobasal thalamus and then on to the cortex, where the sensation of pain is generated.

Intense acute pain and chronic pain may involve the same pathways driven by pathophysiological processes and as such cease to provide a protective mechanism and instead contribute to debilitating symptoms associated with a wide range of disease states. Pain is a feature of many trauma and disease states. When a substantial injury, via disease or trauma, to body tissue occurs the characteristics of nociceptor activation are altered. There is sensitisation in the periphery, locally around the injury and centrally where the nociceptors terminate. This leads to hypersensitivity at the site of damage and in nearby normal tissue. In acute pain these mechanisms can be useful and allow for the repair processes to take place and the hypersensitivity returns to normal once the injury has healed. However, in many chronic pain states, the hypersensitivity far outlasts the healing process and is normally due to nervous system injury. This injury often leads to maladaptation of the afferent fibres (Woolf & Salter 2000 Science 288: 1765-1768). Clinical pain is present when discomfort and abnormal sensitivity feature among the patient's symptoms. Patients tend to be quite heterogeneous and may present with various pain symptoms. There are a number of typical pain subtypes: 1) spontaneous pain which may be dull, burning, or stabbing; 2) pain responses to noxious stimuli are exaggerated (hyperalgesia); 3) pain is produced by normally innocuous stimuli (allodynia) (Meyer et al., 1994 Textbook of Pain 13-44). Although patients with back pain, arthritis pain, CNS trauma, or neuropathic pain may have similar symptoms, the underlying mechanisms are different and, therefore, may require different treatment strategies. Therefore pain can be divided into a number of different areas because of differing pathophysiology, these include nociceptive, inflammatory, neuropathic pain etc. It should be noted that some types of pain have multiple aetiologies and thus can be classified in more than one area, e.g. Back pain, Cancer pain have both nociceptive and neuropathic components.

Nociceptive pain is induced by tissue injury or by intense stimuli with the potential to cause injury. Pain afferents are activated by transduction of stimuli by nociceptors at the site of injury and sensitise the spinal cord at the level of their termination. This is then relayed up the spinal tracts to the brain where pain is perceived (Meyer et al., 1994 Textbook of Pain 13-44). The activation of nociceptors activates two types of afferent nerve fibres. Myelinated A-delta fibres transmitted rapidly and are responsible for the sharp and stabbing pain sensations, whilst unmyelinated C fibres transmit at a slower rate and convey the dull or aching pain. Moderate to severe acute nociceptive pain is a prominent feature of, but is not limited to pain from strains/sprains, post-operative pain (pain following any type of surgical procedure), posttraumatic pain, burns, myocardial infarction, acute pancreatitis, and renal colic. Also cancer related acute pain syndromes commonly due to therapeutic interactions such as chemotherapy toxicity, immunotherapy, hormonal therapy and radiotherapy. Moderate to severe acute nociceptive pain is a prominent feature of, but is not limited to, cancer pain which may be tumour related pain, (e.g. bone pain, headache and facial pain, viscera pain) or associated with cancer therapy (e.g. postchemotherapy syndromes, chronic postsurgical pain syndromes, post radiation syndromes), back pain which may be due to herniated or ruptured intervertabral discs or abnormalities of the lumber facet joints, sacroiliac joints, paraspinal muscles or the posterior longitudinal ligament

Neuropathic pain is defined as pain initiated or caused by a primary lesion or dysfunction in the nervous system (IASP definition). Nerve damage can be caused by trauma and disease and thus the term ‘neuropathic pain’ encompasses many disorders with diverse aetiologies. These include but are not limited to, Diabetic neuropathy, Post herpetic neuralgia, Back pain, Cancer neuropathy, HIV neuropathy, Phantom limb pain, Carpal Tunnel Syndrome, chronic alcoholism, hypothyroidism, trigeminal neuralgia, uremia, or vitamin deficiencies. Neuropathic pain is pathological as it has no protective role. It is often present well after the original cause has dissipated, commonly lasting for years, significantly decreasing a patients quality of life (Woolf and Mannion 1999 Lancet 353: 1959-1964). The symptoms of neuropathic pain are difficult to treat, as they are often heterogeneous even between patients with the same disease (Woolf & Decosterd 1999 Pain Supp. 6: S141-S147; Woolf and Mannion 1999 Lancet 353: 1959-1964). They include spontaneous pain, which can be continuous, or paroxysmal and abnormal evoked pain, such as hyperalgesia (increased sensitivity to a noxious stimulus) and allodynia (sensitivity to a normally innocuous stimulus).

The inflammatory process is a complex series of biochemical and cellular events activated in response to tissue injury or the presence of foreign substances, which result in swelling and pain (Levine and Taiwo 1994: Textbook of Pain 45-56). Arthritic pain makes up the majority of the inflammatory pain population. Rheumatoid disease is one of the commonest chronic inflammatory conditions in developed countries and rheumatoid arthritis is a common cause of disability. The exact aetiology of RA is unknown, but current hypotheses suggest that both genetic and microbiological factors may be important (Grennan & Jayson 1994 Textbook of Pain 397-407). It has been estimated that almost 16 million Americans have symptomatic osteoarthritis (OA) or degenerative joint disease, most of whom are over 60 years of age, and this is expected to increase to 40 million as the age of the population increases, making this a public health problem of enormous magnitude (Houge & Mersfelder 2002 Ann Pharmacother. 36: 679-686; McCarthy et al., 1994 Textbook of Pain 387-395). Most patients with OA seek medical attention because of pain. Arthritis has a significant impact on psychosocial and physical function and is known to be the leading cause of disability in later life. Other types of inflammatory pain include but are not limited to inflammatory bowel diseases (IBD),

Other types of pain include but are not limited to;
Musculo-skeletal disorders including but not limited to myalgia, fibromyalgia, spondylitis, sero-negative (non-rheumatoid) arthropathies, non-articular rheumatism, dystrophinopathy, Glycogenolysis, polymyositis, pyomyositis.
Central pain or ‘thalamic pain’ as defined by pain caused by lesion or dysfunction of the nervous system including but not limited to central post-stroke pain, multiple sclerosis, spinal cord injury, Parkinson's disease and epilepsy.
Heart and vascular pain including but not limited to angina, myocardical infarction, mitral stenosis, pericarditis, Raynaud's phenomenon, scleredoma, scleredoma, skeletal muscle ischemia.
Visceral pain, and gastrointestinal disorders. The viscera encompasses the organs of the abdominal cavity. These organs include the sex organs, spleen and part of the digestive system. Pain associated with the viscera can be divided into digestive visceral pain and non-digestive visceral pain. Commonly encountered gastrointestinal (GI) disorders include the functional bowel disorders (FBD) and the inflammatory bowel diseases (IBD). These GI disorders include a wide range of disease states that are currently only moderately controlled, including—for FBD, gastro-esophageal reflux, dyspepsia, the irritable bowel syndrome (IBS) and functional abdominal pain syndrome MAPS), and—for IBD, Crohn's disease, ileitis, and ulcerative colitis, which all regularly produce visceral pain. Other types of visceral pain include the pain associated with dysmenorrhea, pelvic pain, cystitis and pancreatitis.
Head pain including but not limited to migraine, migraine with aura, migraine without aura cluster headache, tension-type headache.
Orofacial pain including but not limited to dental pain, temporomandibular myofascial pain.

As a yet further aspect, there is provided the use of an alpha-2-delta ligand and an atypical antipsychotic in the manufacture of a medicament for the curative, prophylactic or palliative treatment of pain, particularly neuropathic pain.

As an alternative feature, the invention provides the use of a synergistic effective amount of an alpha-2-delta ligand and an atypical antipsychotic in the manufacture of a medicament for the curative, prophylactic or palliative treatment of pain, particularly neuropathic pain.

As an alternative aspect, there is provided a method for the curative, prophylactic or palliative treatment of pain, particularly neuropathic pain, comprising simultaneous, sequential or separate administration of a therapeutically effective amount of an alpha-2-delta ligand and an atypical antipsychotic, to a mammal in need of said treatment.

As an alternative feature, there is provided a method for the curative, prophylactic or palliative treatment of pain, particularly neuropathic pain, comprising simultaneous, sequential or separate administration of a therapeutically synergistic amount of an alpha-2-delta ligand and an atypical antipsychotic, to a mammal in need of said treatment.

The biological activity of the alpha-2-delta ligands of the invention may be measured in a radioligand binding assay using [³H]gabapentin and the α₂δ subunit derived from porcine brain tissue (Gee N. S., Brown J. P., Dissanayake V. U. K., Offord J., Thurlow R., Woodruff G. N., J. Biol. Chem., 1996; 271:5879-5776). Results may be expressed in terms of μM or nM α2δ binding affinity.

The ability of compounds of the invention to act as atypical antipsychotics can be measured according to established procedures, particularly those described in the documents mentioned hereinabove.

The elements of the combination of the instant invention may be administered separately, simultaneously or sequentially for the treatment of pain. The combination may also optionally be administered with one or more other pharmacologically active agents. Suitable optional agents include:
(i) opioid analgesics, e.g. morphine, heroin, hydromorphone, oxymorphone, levorphanol, levallorphan, methadone, meperidine, fentanyl, cocaine, codeine, dihydrocodeine, oxycodone, hydrocodone, propoxyphene, nalmefene, nalorphine, naloxone, naltrexone, buprenorphine, butorphanol, nalbuphine and pentazocine;
(ii) nonsteroidal antiinflammatory drugs (NSAIDs), e.g. aspirin, diclofenac, diflusinal, etodolac, fenbufen, fenoprofen, flufenisal, flurbiprofen, ibuprofen, indomethacin, ketoprofen, ketorolac, meclofenamic acid, mefenamic acid, nabumetone, naproxen, oxaprozin, phenylbutazone, piroxicam, sulindac, tolmetin, zomepirac, and their pharmaceutically acceptable salts;
(iii) barbiturate sedatives, e.g. amobarbital, aprobarbital, butabarbital, butabital, mephobarbital, metharbital, methohexital, pentobarbital, phenobartital, secobarbital, talbutal, theamylal, thiopental and their pharmaceutically acceptable salts;
(iv) benzodiazepines having a sedative action, e.g. chlordiazepoxide, clorazepate, diazepam, flurazepam, lorazepam, oxazepam, temazepam, triazolam and their pharmaceutically acceptable salts,
(v) H₁ antagonists having a sedative action, e.g. diphenhydramine, pyrilamine, promethazine, chlorpheniramine, chlorcyclizine and their pharmaceutically acceptable salts;
(vi) miscellaneous sedatives such as glutethimide, meprobamate, methaqualone, dichloralphenazone and their pharmaceutically acceptable salts;
(vii) skeletal muscle relaxants, e.g. baclofen, carisoprodol, chlorzoxazone, cyclobenzaprine, methocarbamol, orphrenadine and their pharmaceutically acceptable salts,
(viii) NMDA receptor antagonists, e.g. dextromethorphan ((+)-3-hydroxy-N-methylmorphinan) and its metabolite dextrorphan ((+)-3-hydroxy-N-methylmorphinan), ketamine, memantine, pyrroloquinoline quinone and cis-4-(phosphonomethyl)-2-piperidinecarboxylic acid and their pharmaceutically acceptable salts;
(ix) alpha-adrenergic active compounds, e.g. doxazosin, tamsulosin, clonidine and 4-amino-6,7-dimethoxy-2-(5-methanesulfonamido-1,2,3,4-tetrahydroisoquinol-2-yl)-5-(2-pyridyl)quinazoline;
(x) tricyclic antidepressants, e.g. desipramine, imipramine, amytriptiline and nortriptiline;
(xi) anticonvulsants, e.g. carbamazepine and valproate;
(xii) Tachykinin (NK) antagonists, particularly Nk-3, NK-2 and NK-1 e.g. antagonists, (αR,9R)-7-[3,5-bis(trifluoromethyl)benzyl]-8,9,10,11-tetrahydro-9-methyl-5-(4-methylphenyl)-7H-[1,4]diazocino[2,1-g][1,7]naphthridine-6-13-dione (TAK-637), 5-[[(2R,3S)-2-[(1R)-1-[3,5-bis(trifluoromethyl)phenyl]ethoxy-3-(4-fluorophenyl)-4-morpholinyl]methyl]-1,2-dihydro-3H-1,2,4-triazol-3-one (MK-869), lanepitant, dapitant and 3-[[2-methoxy-5-(trifluoromethoxy)phenyl]methylamino]-2-phenyl-piperidine (2S,3S)
(xiii) Muscarinic antagonists, e.g oxybutin, tolterodine, propiverine, tropsium chloride and darifenacin;
(xiv) COX-2 inhibitors, e.g. celecoxib, rofecoxib and valdecoxib;
(xv) Non-selective COX inhibitors (preferably with GI protection), e.g. nitroflurbiprofen (HCT-1026);
(xvi) coal-tar analgesics, in particular, paracetamol;
(xvii) neuroleptics, such as droperidol;
(xviii) Vanilloid receptor agonists, e.g. resinferatoxin;
(xix) Beta-adrenergic compounds such as propranolol;
(xx) Local anaesthetics, such as mexiletine;
(xxi) Corticosteriods, such as dexamethasone
(xxii) serotonin receptor agonists and antagonists;
(xxiii) cholinergic (nicotinic) analgesics;
(xxiv) miscellaneous agents such as Tramadol®;
(xxv) PDEV inhibitors, such as sildenafil, vardenafil or taladafil;
(xxvi) serotonin reuptake inhibitors, e.g. fluoxetine, paroxetine, citalopram and sertraline;
(xxvii) mixed serotonin-noradrenaline reuptake inhibitors, e.g. milnacipran, venlafaxine and duloxetine;
(xxviii) noradrenaline reuptake inhibitors , e.g. reboxetine.

The present invention extends to a product comprising an alpha-2-delta ligand, an atypical antipsychotic and one or more other therapeutic agents, such as those listed above, for simultaneous, separate or sequential use in the curative, prophylactic treatment of pain, particularly neuropathic pain.

The combination of the invention can be administered alone but one or both elements will generally be administered in an admixture with suitable pharmaceutical excipient(s), diluent(s) or carrier(s) selected with regard to the intended route of administration and standard pharmaceutical practice. If appropriate, auxiliaries can be added. Auxiliaries are preservatives, anti-oxidants, flavours or colourants. The compounds of the invention may be of immediate-, delayed-, modified-, sustained-, pulsed- or controlled-release type.

The elements of the combination of the present invention can be administered, for example but not limited to, the following route: orally, buccally or sublingually in the form of tablets, capsules, multi-and nano-particulates, gels, films (incl. muco-adhesive), powder, ovules, elixirs, lozenges (incl. liquid-filled), chews, solutions, suspensions and sprays. The compounds of the invention may also be administered as osmotic dosage form, or in the form of a high energy dispersion or as coated particles or fast-dissolving, fast-disintegrating dosage form as described in Ashley Publications, 2001 by Liang and Chen. The compounds of the invention may be administered as crystalline or amorphous products, freeze dried or spray dried. Suitable formulations of the compounds of the invention may be in hydrophilic or hydrophobic matrix, ion-exchange resin complex, coated or uncoated form and other types as described in U.S. Pat. No. 6,106,864 as desired.

Such pharmaceutical compositions, for example, tablets, may contain excipients such as microcrystalline cellulose, lactose, sodium citrate, calcium carbonate, dibasic calcium phosphate, glycine and starch (preferably corn, potato or tapioca starch), mannitol, disintegrants such as sodium starch glycolate, crosscarmellose sodium and certain complex silicates, and granulation binders such as polyvinylpyrrolidone, hydroxypropylmethylcellulose (HPMC), triglycerides, hydroxypropylcellulose (HPC), bentonite sucrose, sorbitol, gelatin and acacia. Additionally, lubricating agents may be added to solid compositions such as magnesium stearate, stearic acid, glyceryl behenate, PEG and talc or wetting agents, such as sodium lauryl sulphate. Additionally, polymers such as carbohydrates, phospoholipids and proteins may be included.

Fast dispersing or dissolving dosage fromulations (FDDFs) may contain the following ingredients: aspartame, acesulfame potassium, citric acid, croscarmellose sodium, crospovidone, diascorbic acid, ethyl acrylate, ethyl cellulose, gelatin, hydroxypropylmethyl cellulose, magnesium stearate, mannitol, methyl methacrylate, mint flavouring, polyethylene glycol, fumed silica, silicon dioxide, sodium starch glycolate, sodium stearyl fumarate, sorbitol or xylitol. The terms dispersing or dissolving as used herein to describe FDDFs are dependent upon the solubility of the drug substance used, i.e. where the drug substance is insoluble a fast dispersing dosage form can be prepared and where the drug substance is soluble a fast dissolving dosage form can be prepared.

The solid dosage form, such as tablets are manufactured by a standard process, for example, direct compression or a wet, dry or melt granulation, melt congealing and extrusion process. The tablet cores which may be mono or multi-layer may be coated with appropriate overcoats known in the art.

Solid compositions of a similar type may also be employed as fillers in capsules such as gelatin, starch or HPMC capsules. Preferred excipients in this regard include lactose, starch, a cellulose, milk sugar or high molecular weight polyethylene glycols. Liquid compositions may be employed as fillers in soft or hard capsules such as gelatin capsule. For aqueous and oily suspensions, solutions, syrups and/or elixirs, the compounds of the invention may be combined with various sweetening or flavouring agents, colouring matter or dyes, with emulsifying and/or suspending agents and with diluents such as water, ethanol, propylene glycol, methylcellulose, alginic acid or sodium alginate, glycerin, oils, hydrocolloid agents and combinations thereof. Moreover, formulations containing these compounds and excipients may be presented as a dry product for constitution with water or other suitable vehicles before use.

Liquid form preparations include solutions, suspensions, and emulsions, for example, water or water propylene glycol solutions. For parenteral injection, liquid preparations can be formulated in solution in aqueous polyethylene glycol solution. Aqueous solutions suitable for oral use can be prepared by dissolving the active component in water and adding suitable colorants, flavors, stabilizing and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

The elements of the combination of the present invention can also be administered by injection, that is, intravenously, intramuscularly, intracutaneously, intraduodenally, or intraperitoneally, intraarterially, intrathecally, intraventricularly, intraurethrally, intrasternally, intracranially, intraspinally or subcutaneously, or they may be administered by infusion, needle-free injectors or implant injection techniques. For such parenteral administration they are best used in the form of a sterile aqueous solution, suspension or emulsion (or system so that can include micelles) which may contain other substances known in the art, for example, enough salts or carbohydrates such as glucose to make the solution isotonic with blood. The aqueous solutions should be suitably buffered (preferably to a pH of from 3 to 9), if necessary. For some forms of parenteral administration they may be used in the form of a sterile non-aqueous system such as fixed oils, including mono- or diglycerides, and fatty acids, including oleic acid. The preparation of suitable parenteral formulations under sterile conditions for example lyophilisation is readily accomplished by standard pharmaceutical techniques well-known to those skilled in the art. Alternatively, the active ingredient may be in powder form for constitution with a suitable vehicle (e.g. sterile, pyrogen-free water) before use.

Also, the elements of the combination of the present invention can be administered intranasally or by inhalation. They are conveniently delivered in the form of a dry powder (either alone, as a mixture, for example a dry blend with lactose, or a mixed component particle, for example with phospholipids) from a dry powder inhaler or an aerosol spray presentation from a pressurised container, pump, spray, atomiser (preferably an atomiser using electrohydrodynamics to produce a fine mist) or nebuliser, with or without the use of a suitable propellant, e.g. dichlorodifluoromethane, trichlorofluoromethane, dichlorotetrafluoroethane, a hydrofluoroalkane such as 1,1,1,2-tetrafluoroethane (HFA 134A [trade mark]) or 1,1,1,2,3,3,3-heptafluoropropane (HFA 227EA [trade mark]), carbon dioxide, a further perfluorinated hydrocarbon such as Perflubron (trade mark) or other suitable gas. In the case of a pressurised aerosol, the dosage unit may be determined by providing a valve to deliver a metered amount. The pressurised container, pump, spray, atomiser or nebuliser may contain a solution or suspension of the active compound, e.g. using a mixture of ethanol (optionally, aqueous ethanol) or a suitable agent for dispersing, solubilising or extending release and the propellant as the solvent, which may additionally contain a lubricant, e.g. sorbitan trioleate. Capsules, blisters and cartridges (made, for example, from gelatin or HPMC) for use in an inhaler or insufflator may be formulated to contain a powder mix of the compound of the invention, a suitable powder base such as lactose or starch and a performance modifier such as 1-leucine, mannitol or magnesium stearate.

Prior to use in a dry powder formulation or suspension formulation for inhalation the elements of the combination of the invention will be micronised to a size suitable for delivery by inhalation (typically considered as less than 5 microns). Micronisation could be achieved by a range of methods, for example spiral jet milling, fluid bed jet milling, use of supercritical fluid crystallisation or by spray drying.

A suitable solution formulation for use in an atomiser using electrohydrodynamics to produce a fine mist may contain from 1 μg to 10 mg of the compound of the invention per actuation and the actuation volume may vary from 1 to 100 μl. A typical formulation may comprise the elements of the combination of the invention, propylene glycol, sterile water, ethanol and sodium chloride. Alternative solvents may be used in place of propylene glycol, for example glycerol or polyethylene glycol.

Alternatively, the elements of the combination of the invention may be administered topically to the skin, mucosa, dermally or transdermally, for example, in the form of a gel, hydrogel, lotion, solution, cream, ointment, dusting powder, dressing, foam, film, skin patch, wafers, implant, sponges, fibres, bandage, microemulsions and combinations thereof. For such applications, the compounds of the invention can be suspended or dissolved in, for example, a mixture with one or more of the following: mineral oil, liquid petrolatum, white petrolatum, propylene glycol, polyoxyethylene polyoxypropylene compound, emulsifying wax, fixed oils, including synthetic mono- or diglycerides, and fatty acids, including oleic acid, water, sorbitan monostearate, a polyethylene glycol, liquid paraffin, polysorbate 60, cetyl esters wax, cetearyl alcohol, 2-octyldodecanol, benzyl alcohol, alcohols such as ethanol. Alternatively, penetration enhancers may be used. The following may also be used; polymers, carbohydrates, proteins, phospolipids in the form of nanoparticles (such as niosomes or liposomes) or suspended or dissolved. In addition, they may be delivered using iontophoresis, electroporation, phonophoresis and sonophoresis.

Alternatively, the elements of the combination of the invention can be administered rectally, for example in the form of a suppository or pessary. They may also be administered by vaginal route. For example, these compositions may be prepared by mixing the drug with suitable non-irritant excipients, such as cocoa butter, synthetic glyceride esters or polyethylene glycols, which are solid at ordinary temperatures, but liquefy and/or dissolve in the cavity to release the drug.

The elements of the combination of the invention may also be administered by the ocular route. For ophthalmic use, the compounds can be formulated as micronised suspensions in isotonic, pH adjusted, sterile saline, or, preferably, as solutions in isotonic, pH adjusted, sterile saline. A polymer may be added such as crossed-linked polyacrylic acid, polyvinylalcohol, hyaluronic acid, a cellulosic polymer (e.g. hydroxypropylmethylcellulose, hydroxyethylcellulose, methyl cellulose), or a heteropolysaccharide polymer (e.g. gelan gum). Alternatively, they may be formulated in an ointment such as petrolatum or mineral oil, incorporated into biodegradable (e.g. absorbable gel sponges, collagen) or non-biodegradable (e.g. silicone) implants, wafers, drops, lenses or delivered via particulate or vesicular systems such as niosomes or liposomes. Formulations may be optionally combined with a preservative, such as benzalkonium chloride. In addition, they may be delivered using iontophoresis. They may also be administered in the ear, using for example but not limited to the drops.

The elements of the combination of the invention may also be used in combination with a cyclodextrin. Cyclodextrins are known to form inclusion and non-inclusion complexes with drug molecules. Formation of a drug-cyclodextrin complex may modify the solubility, dissolution rate, taste-masking, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta- and gamma-cyclodextrins are most commonly used and suitable examples are described in WO-A-91/11172, WO-A-94/02518 and WO-A-98/55148.

The term ‘administered’ includes delivery by viral or non-viral techniques. Viral delivery mechanisms include but are not limited to adenoviral vectors, adeno-associated viral (AAV) vectors, herpes viral vectors, retroviral vectors, lentiviral vectors, and baculoviral vectors. Non-viral delivery mechanisms include lipid mediated transfection, lipsomes, immunoliposomes, lipofectin, cationic facial amphiphiles (CFAs) and combinations thereof. The routes for such delivery mechanisms include but are not limited to mucosal, nasal, oral, parenteral, gastrointestinal, topical or sublingual routes.

Thus, as a further aspect of the present invention, there is provided a pharmaceutical composition comprising a combination comprising an alpha-2-delta ligand, an atypical antipsychotic, or pharmaceutically acceptable salts thereof, and a suitable excipient, diluent or carrier. Suitably, the composition is suitable for use in the treatment of pain, particularly neuropathic pain.

As an alternative aspect of the present invention, there is provided a pharmaceutical composition comprising a synergistic combination comprising an alpha-2-delta ligand, an atypical antipsychotic, or pharmaceutically acceptable salts thereof, and a suitable excipient, diluent or carrier. Suitably, the composition is suitable for use in the treatment of pain, particularly neuropathic pain.

For non-human animal administration, the term ‘pharmaceutical’ as used herein may be replaced by ‘veterinary.’

The element of the pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsules, tablet, cachet, or lozenge itself, or it can be the appropriate number of any of these in packaged form. The quantity of active component in a unit dose preparation may be varied or adjusted from 0.1 mg to 1 g according to the particular application and the potency of the active components. In medical use the drug may be administered three times daily as, for example, capsules of 100 or 300 mg. In therapeutic use, the compounds utilized in the pharmaceutical method of this invention are administered at the initial dosage of about 0.01 mg to about 100 mg/kg daily. A daily dose range of about 0.01 mg to about 100 mg/kg is preferred. The dosages, however, may be varied depending upon the requirements of the patient, the severity of the condition being treated, and the compounds being employed. Determination of the proper dosage for a particular situation is within the skill of the art. Generally, treatment is initiated with smaller dosages which are less than the optimum dose of the compounds. Thereafter, the dosage is increased by small increments until the optimum effect under the circumstances is reached. For convenience, the total daily dosage may be divided and administered in portions during the day, if desired.

For veterinary use, a combination according to the present invention or veterinarily acceptable salts or solvates thereof, is administered as a suitably acceptable formulation in accordance with normal veterinary practice and the veterinary surgeon will determine the dosing regimen and route of administration which will be most appropriate for a particular animal.

### BIOLOGY EXAMPLES

Methods

Animals

Male Sprague Dawley rats (200-250 g), obtained from Charles River, (Margate, Kent, U.K.) are housed in groups of 6. All animals are kept under a 12 h light/dark cycle (lights on at 07 h 00 min) with food and water ad libitum. All experiments are carried out by an observer unaware of drug treatments.

CCI Surgery in the Rat

Animals are anaesthetised with isoflurane. The sciatic nerve is ligated as previously described by Bennett and Xie, 1988. Animals are placed on a homeothermic blanket for the duration of the procedure. After surgical preparation the common sciatic nerve is exposed at the middle of the thigh by blunt dissection through biceps femoris. Proximal to the sciatic trifurcation, about 7 mm of nerve is freed of adhering tissue and 4 ligatures (4-0 silk) are tied loosely around it with about 1 mm spacing. The incision is closed in layers and the wound treated with topical antibiotics.

Effect of Combinations on the Maintenance of CCI-Induced Static and Dynamic Allodynia

Dose-responses to gabapentin and an atypical antipsychotic are first performed alone in the CCI model. Combinations are examined following a fixed ratio design. A dose-response to each fixed dose ratio of the combination is performed. On each test day, baseline paw withdrawal thresholds (PWT) to von Frey hairs and paw withdrawal latencies (PWL) to a cotton bud stimulus are determined prior to drug treatment.

Evaluation of Allodynia

Static allodynia is measured using Semmes-Weinstein von Frey hairs (Stoelting, Ill., U.S.A.). Animals are placed into wire mesh bottom cages allowing access to the underside of their paws. Animals are habituated to this environment prior to the start of the experiment. Static allodynia is tested by touching the plantar surface of the animals right hind paw with von Frey hairs in ascending order of force (0.7, 1.2, 1.5, 2, 3.6, 5.5, 8.5, 11.8, 15.1 and 29 g) for up to 6 sec. Once a withdrawal response is established, the paw is re-tested, starting with the next descending von Frey hair until no response occurs. The highest force required to lift the paw as well as elicit a response, thus represents the cut off point. The lowest amount of force required to elicit a response is recorded as the PWT in grams.

Dynamic allodynia is assessed by lightly stroking the plantar surface of the hind paw with a cotton bud. Care is taken to perform this procedure in fully habituated rats that are not active to avoid recording general motor activity. At least three measurements are taken at each time point the mean of which represents the paw withdrawal latency (PWL). If no reaction is exhibited within 15 s the procedure is terminated and animals are assigned this withdrawal time. Thus 15 s effectively represents no withdrawal. A withdrawal response is often accompanied with repeated flinching or licking of the paw. Dynamic allodynia is considered to be present if animals responded to the cotton stimulus before 8 s of stroking.

Combination Studies

Dose responses are first performed to both the alpha-2-delta ligand (p.o.) and atypical antipsychotic (s.c. or p.o.) alone. A number of fixed dose ratios of the combination may then be examined. Dose responses to each fixed dose ratio are performed with the time-course for each experiment determined by the duration of antiallodynic-action of each separate ratio. Various fixed dose ratios of the combinations by weight may be examined.

Suitable atypical antipsychotic compounds of the present invention may be prepared as described in the references or are obvious to those skilled in the art on the basis of these documents.

Suitable alpha-2-delta ligand compounds of the present invention may be prepared as described herein below or in the aforementioned patent literature references, which are illustrated by the following non-limiting examples and intermediates.

The following examples and preparations illustrate the preparation of atypical antipsychotics disclosed in PCT/IB2004/002985:

### Example 1

### (S)-3-((E)-2-Methyl-pent-2-enoyl)-4-phenyl-oxazolidin-2-one

A 20 L jacketed reactor was fitted with a reflux condenser and a nitrogen inlet. To the flask was charged 1006 g (8.81 mol) of (E)-2-methyl-2-pentenoic acid, 1250 g (7.661 mol) of (S)-(+)-4-phenyl-oxazolidin-2-one, 2179 g (8.81 mol) of 2-ethoxy-1-ethoxycarbonyl-1,2-dihydroquinoline (EEDQ), 81 g (1.915 mol) of lithium chloride, and 12.5 L of ethyl acetate (EtOAc). The reaction was heated to 75□C for 20 hours and then cooled to room temperature. The reaction solution was extracted 3× with 4 L aliquots of 1N HCl and 1× with 4 L of 0.2N NaOH. The 20 L reactor was fitted with a distillation head. The organic layer was distilled to remove, in succession: 6.5 L of EtOAc, after which 8 L of heptane was added back to the reactor; 4 L of EtOAc/heptane, after which 4 L of heptane was added to the reactor; and 4 L of EtOAc/heptane, after which 8 L of heptane was added to the reactor. After an additional 2 L of EtOAc/heptane was removed by distillation, the reaction mixture was cooled to an internal temperature of 40° C., and the reactor contents were charged to a filter and filtered under 5 psig of nitrogen washing with 8 L of heptane. The solids were dried under 5 psig of nitrogen overnight to give 1772 g of the titled compound: ¹H-NMR (DMSO), 7.363-7.243 (m, 5H), 6.137-6.096 (m, 1H), 5.434-5.394 (m, 1H), 4.721-4.678 (t, 1H, J=8.578), 4.109-4.069 (m, 1H), 2.119-2.044 (m, 2H), 1.703-1.700 (d, 3H, J=1.364), 0.945-0.907 (t, 3H, J=7.603); Anal Calc'd for C₁₅H₁₇N₁O₃: C, 69.48; H, 6.61; N, 5.40. Found: C, 68-66; H, 6.60; N, 5.60; MS (Ion Mode: APCI), m/z=260 [M+1]⁺.

### (4S,5R)-3-((E)-2-Methyl-pent-2-enoyl)-4,5-diphenyl-oxazolidin-2-one

To a solution of (E)-2-methyl-2-pentenoic acid (5.3 g, 47 mmol) in 250 mL of THF at 0° C. was added 16.3 mL (117 mmol) of triethylamine, then 5.8 mL (47 mmol) of pivaloyl chloride resulting in a thick suspension. The mixture was stirred for 1 hour at 0° C. at which time 2.0 g (47 mmol) of lithium chloride was added in one portion, followed by 10 g (42 mmol) of (4S,5R)-4,5-diphenyl-2-oxazolidinone in four batches. Stirring was maintained throughout the solid additions. The reaction mixture was stirred for 1 hour at 0° C., and for 1 hour at ambient temperature, and was vacuum filtered through a coarse frit and concentrated. The residue was partitioned between EtOAc/water, and the organics were dried over MgSO₄ and concentrated. To the residue was added 200 mL of MTBE and the mixture was warmed cautiously with swirling. The warm slurry was filtered to provide 13.0 g (83% yield) of the titled compound as a colorless solid: ¹H NMR (CDCl₃), δ 7.12 (m, 3H), 7.08 (m, 3H), 6.93 (m, 2H), 6.86 (m, 2H), 6.14 (m, 1H), 5.90 (d, J=7.8 Hz, 1H), 5.69 (d, J=7.8 Hz, 1H), 2.23 (pent, J=7.6 Hz, 2H), 1.92 (s, 3H), 1.07 (t, J=7.6 Hz, 3H). The titled acylated oxazolidinone may be used in the next step instead of (S)-3-((E)-2-Methyl-pent-2-enoyl)-4-phenyl-oxazolidin-2-one.

### (2R,3R,4S)-3-(2,3-Dimethyl-pentanoyl)-4-phenyl-oxazolidin-2-one

A 20 L jacketed reactor was fit with a gas inlet and a 2 L dripping funnel. A nitrogen sweep was begun over the reactor and maintained throughout the process. To the reactor was charged 392 g (9.26 mol) of lithium chloride, 1332 g (6.479 mol) of copper bromide dimethylsulfide complex and 11 L of tetrahydrofuran. The reaction was stirred for 30 minutes at room temperature and then cooled to −15° C. To the reaction mixture was added 4.268 L (12.80 mol) of 3.0M methyl magnesium chloride at a rate such that the reaction temperature did not exceed −10° C. Upon completion of the addition, the cuprate solution was allowed to stir at −5° C. overnight. To the cuprate solution was added 500 g (3.09 mol) of (S)-3-((E)-2-methyl-pent-2-enoyl)-4-phenyl-oxazolidin-2-one as a solid. The reaction was stirred at −3□C for 2 hours. The reaction solution was charged to a 22 L round bottom flask containing 800 mL of acetic acid and 2 L of tetrahydrofuran at a rate such that the temperature of the quench solution did not exceed 25° C. To the quenched solution was added 6 L water. The resulting emulsion was filtered and the layers were separated. The organic layer was extracted with 9 L of 4.8 M NH₄OH followed by 9 L of saturated NH₄Cl. The organic layer was clarified through a plug of magnesol. The organic layer was concentrated to give 822 g of a crude solid. The crude solid was recrystallized from 8 L of 20% H₂O in MeOH, filtered and dried in a vacuum oven to give 550 g of a white solid. The white solid was recrystallized from 5 L of 20% H₂O in MeOH, filtered and dried in a vacuum oven to give 475 g of the titled compound: ¹H-NMR (DMSO), 7.338-7.224 (m, 5H), 5.431-5.399 (q, 1H, J=4.288), 4.696-4.652 (t, 1H, J=8.773), 4.120-4.087 (m, 1H), 3.622-3.556 (m, 1H), 1.648-1.584 (m, 1H), 1.047-0.968 (m, 1H1), 0.900-0.883 (d, 3H, J=6.823), 0.738-0.721 (d, 3H, J=6.628), 0.693-0.656 (t, 3H, J=7.408); Anal Calc'd for C₁₆H₂₁N₁O₃: C, 69.79; H, 7.69; N, 5.09. Found: C, 69.81; H, 7.61; N, 5.07; MS (Ion Mode: APCI), m/z=276 [M+1]⁺.

### (2R,3R)-2,3-Dimethyl-pentanoic acid

A 20 L jacketed flask was fit with a gas inlet. A nitrogen purge was begun over the reactor and maintained throughout the process. To the flask was charged 450 g (1.634 mol) of (2R,3R,4S)-3-(2,3-dimethyl-pentanoyl)-4-phenyl-oxazolidin-2-one and 3.375 L tetrahydrofuran. The contents of the reactor were stirred at 15° C. In a separate 3 L round bottom flask, placed in an ice bath, was charged 500 mL of water, 137 g (3.269 mol) of LiOH—H₂O and 942 mL (9.81 mol) of 30% wt/wt H₂O₂. The contents of the 3 L round bottom flask were stirred for 3 minutes and then poured into the 20 L jacketed reactor at a rate such that the temperature did not exceed 25° C. The reaction was stirred at 15° C. for 2 hours and then raised to 25° C. and stirred for an additional 2 hours. The jacket temperature of the reactor was set to −20° C. To the reaction was added 1.66 L of saturated NaHSO₃ at a rate such that the temperature of the reaction did not exceed 25° C. The layers were separated. The aqueous layer was extracted 2× with 1 L aliquots of MTBE. The organic phases were combined and concentrated to give a solid/oil mixture. The solid/oil mixture was slurried in 1.7 L of hexane. The slurry was filtered and the collected solids were washed with 1.7 L of hexane. The hexane filtrates were extracted 2× with 1.35 L aliquots of 1N NaOH. The aqueous extracts were combined and extracted with 800 mL of dichloromethane. The aqueous layer was then acidified with 240 mL of concentrated hydrochloric acid. The aqueous solution was extracted 2× with 1 L aliquots of dichloromethane. The organic extracts were combined, dried over MgSO₄ and concentrated to give 201 g of the titled compound: ¹H-NMR (DMSO), 11.925 (bs, 1H), 2.204-2.135 (m, 1H), 1.556-1.490 (m, 1H), 1.382-1.300 (m, 1H), 1.111-1.000 (m, 1H), 0.952-0.934 (d, 3H, J=7.018), 0.809-0.767 (m, 6H); Gas Chromatogram 9.308 minutes, 98.91% area; Anal Calc'd for C₇H₁₄O₂: C, 64.58; H, 10.84; N, 0. Found: C, 64.39; H, 10.77; N, 0.18; MS (Ion Mode: APCI), m/z=131 [M+1]⁺.

### (4R,5R)-4,5-Dimethyl-3-oxo-heptanoic acid ethyl ester

To a 1 L round bottom flask equipped with a nitrogen inlet was charged 22 g (230 mmol) of magnesium chloride, 39 g (230 mmol) of potassium ethyl malonate and 200 mL of dimethylformamide. The contents of the flask were stirred at 50° C. for 1 hour and then cooled to 35° C. In a separate 500 mL, nitrogen inerted flask was added 200 mL of dimethylformamide, 28.6 g (177 mmol) of carbonyl diimidazole and 20 g of (2R,3R)-2,3-dimethyl-pentanoic acid was dripped in over 30 minutes. When the gas evolution had ceased, the contents of the 500 mL flask were added to the 1 L flask. The reaction was stirred for 2 days at 35° C. The reaction was cooled to room temperature and diluted with 800 mL of 1N HCl. The aqueous solution was extracted 3× with 1 L aliquots of MTBE. The organic extracts were combined and extracted with 200 mL of saturated NaHCO₃. The organic layer was dried over MgSO₄ and concentrated to give 31.74 g of the titled compound: ¹H-NMR (CDCl₃), 4.180-4.120 (m, 2H), 3.454 (s, 2H), 2.522-2.453 (q, 1H, J=7.018), 1.738-1.673 (m, 1H), 1.418-1.328 (m, 1H), 1.270-1.217 (m, 3H), 1.113-1.010 (m, 4H), 0.889-0.815 (m, 5H); MS (Ion Mode: APCI), m/z=201 [M+1]⁺.

### (4R,5R)-3-Methoxyimino-4,5-dimethyl-heptanoic acid ethyl ester

(4R,5R)-4,5-Dimethyl-3-oxo-heptanoic acid ethyl ester (21.23 g, 106 mmol) was dissolved in 200 mL of EtOH and added to 10.6 g (127 mmol) of methoxylamine-HCl and 10.6 g (127 mmol) of sodium acetate solids. The slurry was stirred at room temperature for 48 hours. MTBE (200 mL) and 100 mL of water were added, and the resulting phases were separated. The organic phase was washed with 100 mL of water and was evaporated to yield a two-phase mixture. Hexanes (100 mL) were added and the phases were separated. The aqueous phase was extracted with 50 mL of hexanes and the combined organic phases were washed with 50 mL of water, dried over magnesium sulfate, and evaporated to give 21.24 g (87.4% yield) of the titled compound as a clear yellow oil: ¹H NMR (CDCl₃, 399.77 MHz), δ 0.84-0.88 (m, 6H), 1.07 (d, J=7.1 Hz, 3H), 1.24 (t, J=7.1 Hz, 3H), 1.4-1.6 (m, 2H), 2.24 (m, 1H), 3.08 (d, J=15.8 Hz, 1H), 3.19 (d, J=15.8 Hz, 1H), 3.80 (s, 3H), 4.10-4.2 (m, 3H). Low resolution mass spec: nominal m/e calc'd for C₁₂H₂₃NO₃ (M+H)⁺: 230. Found: m/e 230.

### (4R,5R)-3-Amino-4,5-dimethyl-hept-2-(Z)-enoic acid ethyl ester

A solution of 21.1 g (92 mmol) of (4R,5R)-3-methoxyimino-4,5-dimethyl-heptanoic acid ethyl ester in methanol (200 mL) was treated with Sponge nickel (10 g, Johnson Matthey A7000). The resulting slurry was hydrogenated on a Parr shaker type hydrogenator at 50 psig and room temperature for 20 hours. At this time an additional 10 g of the nickel catalyst was added and hydrogenation was continued for a total of 42.0 hours. The slurry was filtered, the solids were washed with fresh methanol, and the combined filtrate was evaporated to give 17.75 g (96.8% yield) of the titled compound as a colorless oil: ¹H NMR (CDCl₃, 399.77 MHz), δ 0.83-0.89 (m, 6H), 1.1 (d, J=6.8 Hz, 3H), 1.25 (t, J=7.1 Hz, 2H), 1.35-1.6 (m, 4H), 1.85-1.93 (m, 1H), 4.1 (q, J=7.0 Hz, 2H), 4.5 (s, 1H). Low resolution mass spec: nominal m/e calc'd for C₁₁H₂₁NO₂ (M+H)⁺: 200. Found: m/e 200.

### (4R,5R)-3-Acetylamino-4,5-dimethyl-hept-2-(Z)-enoic acid ethyl ester

A solution of 15.84 g (79.84 mmol) of (4R,5R)-3-amino-4,5-dimethyl-hept-2-(Z)-enoic acid ethyl ester and 6.89 g (7.04 mL, 87.82 mL) of pyridine was stirred in 200 mL of methylene chloride and cooled to 0° C. A solution of 6.85 g (6.21 mL, 87.82 mL) of acetyl chloride in 20 mL of methylene chloride was added dropwise over 1 hour. The solution was warmed to room temperature and stirred for two hours. 1M hydrochloric acid (100 mL) was added and the phases were separated. The organic phase was washed with saturated aqueous NaHCO₃ solution and dried briefly over Na₂SO₄. The solvent was evaporated and then the resulting oil was passed through a short column of silica (200 g silica, 230-400 mesh) with 8:1 (v/v) hexane/EtOAc. The product-containing fractions were evaporated to give 13.75 g (71.7% yield) of the titled compound as a clear, nearly colorless oil: ¹H NMR (CDCl₃, 399.77 MHz), δ 0.84 (t, J=7.1 Hz, 3H), 0.95 (d, J=6.8 Hz, 3H), 1.0 (d, J=7.0 Hz, 3H), 1.29 (t, J=7.2 Hz, 3H), 1.30-1.45 (m, 3H), 2.13 (s, 3H), 3.79-3.82 (m, 1H), 4.11-4.18 (m, 2H), 5.01 (s, 1H). Low resolution mass spec: nominal m/e calc'd for C₁₃H₂₃NO₃ (M+H)⁺: 242. Found: m/e 242.

### (3R,4R,5R)-3-Acetylamino-4,5-dimethyl-heptanoic acid ethyl ester

A solution containing 13.75 g (57 mmol) of (4R,5R)-3-acetylamino-4,5-dimethyl-hept-2-(Z)-enoic acid ethyl ester in 200 mL of methanol was treated with 5% Pd/Al₂O₃ (1.5 g, Johnson Matthey #2127, lot 13449). The resulting slurry was hydrogenated on a Parr shaker type hydrogenator at 40 psig to 50 psig and room temperature for a total of 3.8 hours. The slurry was filtered and the solids were washed with fresh methanol. The combined filtrate was evaporated to give 13.63 g (98.6% yield) of the titled compound as a colorless oil: ¹H NMR (CDCl₃, 399.77 MHz), δ 0.82 (d, J=7.0 Hz, 3H), 0.86 (t, J=7.3 Hz, 3H), 0.90 (d, J=6.5 Hz, 3H), 0.98-1.1 (m, 2H), 1.25 (t, J=7.2 Hz, 2H), 1.3-1.6 (m, 2H), 1.96 (s, 3H), 2.48 (dd, J=16, 5.65 Hz, 1H), 2.53 (dd, J=16, 5.2 Hz, 1H), 4.08-4.19 (m, 2H), 4.27-4.34 (m, 1H), 5.86 (br d, J=8.9 Hz, 1H). Low resolution mass spec: nominal m/e calc'd for C₁₃H₂₅NO₃ (M+H)⁺: 244. Found: m/e 244.

### (3R,4R,5R)-3-Amino-4,5-dimethyl-heptanoic acid hydrochloride

(3R,4R,5R)-3-Acetylamino-4,5-dimethyl-heptanoic acid ethyl ester (13.63 g, 56.0 mmol) was heated under reflux with 200 mL of 1M hydrochloric acid for 72 hours. The solution was cooled and extracted 2× with 50 mL aliquots of MTBE. The aqueous phase was evaporated to a semisolid. Acetonitrile (4×100 mL) was added and evaporated to give 10.75 g (89% yield) of the titled compound as a white crystalline solid: ¹H NMR (CD₃OD, 399.77 MHz), 0.87 (t, J=7.3 Hz, 3H), 0.94 (t, J=6.6 Hz, 6H), 1.02-1.15 (m, 1H), 1.37-1.53 (m, 2H), 1.58-1.68 (m, 1H), 2.64 (dd, J=17.5, 7.4 Hz, 1H), 2.73 (dd, J+17.5, 4.8 Hz, 1H), 3.54-3.61 (m, 1H). Low resolution mass spec: nominal m/e calc'd for C₉H₂₀ClNO₂ (M+H)⁺: 174. Found: m/e 174.

### (3R,4R,5R)-3-Amino-4,5-dimethyl-heptanoic acid

(3R,4R,5R)-3-Amino-4,5-dimethyl-heptanoic acid hydrochloride (10.8 g, 51.5 mmol) was dissolved in 50 mL of methanol. To this solution was added triethylamine (5.2 g, 7.2 mL, 51.5 mmol). The solution was stirred for 10 minutes and then evaporated to a flocculent solid. Dichloromethane (376 mL) was added and the resulting slurry was stirred at room temperature for 45 minutes. Next, 188 mL of acetonitrile was added and the slurry was stirred for 30 minutes and then filtered. The solids were washed with 20 mL of 2:1 (v/v) dichloromethane-acetonitrile and dried on a nitrogen press to give 7.64 g (85.6% yield) of the titled compound as a white solid: ¹H NMR (CD₃OD, 399.77 MHz), 0.88 (t, J=7.5 Hz, 3H), 0.91 (d, J=7.0 Hz, 3H), 0.94 (d, J=6.6 Hz, 3H), 0.98-1.12 (m, 1H), 1.32-1.43 (m, 1H), 1.43-1.64 (m, 2H), 2.26 (dd, J=16.5, 9.9 Hz, 1H), 2.47 (dd, J=19.5, 3.7 Hz, 1H), 3.28-3.36 (m, 1H). Low resolution mass spec: nominal m/e calc'd for C₉H₁₉NO₂ (M+H)⁺: 174. Found: m/e 174.

### (3R,4R,5R)-3-Amino-4,5-dimethyl-heptanoic Acid-1/6-succinic acid complex-1/6-hydrate, i.e., 6-((3R,4R,5R)-3-amino-4,5-dimethyl-heptanoic acid): 1-(succinic acid): 1-(H₂O)

(3R,4R,5R)-3-Amino-4,5-dimethyl-heptanoic acid (7.6 g, 44 mmol) and succinic acid (2.6 g, 22 mmol) were suspended in 20.2 mL of water. The slurry was heated to 100° C. to dissolve the solids. Acetonitrile (253 mL) was added to the hot solution. The mixture was stirred at 55° C. for 1 hour, and then cooled gradually to room temperature overnight. The resulting solids were filtered, washed with 10 mL of acetonitrile, and dried on a nitrogen press to give 6.21 g (72% yield) of the titled compound as fluffy white crystals: ¹H NMR (CD₃OD, 399.77 MHz), ¹H NMR (CD₃OD, 399.77 MHz), 0.88 (t, J=7.5 Hz, 3H), 0.91 (d, J=7.0 Hz, 3H), 0.94 (d, J=6.6 Hz, 3H), 0.98-1.12 (m, 1H), 1.32-1.43 (m, 1H), 1.43-1.64 (m, 2H), 2.26 (dd, J=16.5, 9.9 Hz, 1H), 2.47 (dd, J=19.5, 3.7 Hz, 1H), 2.50 (s, 0.67H), 3.28-3.36 (m, 1H). Low resolution mass spec: nominal m/e calc'd for C₉H₁₉NO₂(M+H)⁺: 174. Found: m/e 174. Anal. calc'd for 6-((3S,4R,5R 3-amino-4,5-dimethyl-heptanoic Acid):1-(succinic Acid):1-(H₂O), C₅₈H₁₂₂N₆O₁₃: C, 59.26; H, 10.46; N, 7.15. Found: C, 59.28; H, 10.58; N, 7.09. KF calc'd for C₅₈H₁₂₂N₆O₁₃:H₂O, 1.43 wt %. Found: H₂O, 1.50 wt %.

### Example 2

### (4S,5R)-4,5-Diphenyl-oxazolidin-2-one

To a 5 L round bottom flask equipped with an overhead stirrer, thermocouple and distillation head, was charged 550 g (2.579 mol) of (1R,2S)-diphenyl-2-aminoethanol, 457 g (3.868 mol, 1.5 eq) of diethylcarbonate, 18 g (0.258 mol, 0.1 eq) of NaOEt in 100 mL of EtOH and 3.5 L of toluene. The reaction was heated until an internal temperature of 90° C. was reached and EtOH distillation began. The reaction was refluxed until an internal temperature of 110□C was reached (7 hours). For every 500 mL of solvent that was removed via the distillation head, 500 mL of toluene was added back to the reaction. A total of about 1.6 L of solvent was removed. The reaction was allowed to cool to room temperature and then filtered on a 3 L coarse fritted funnel with 2 psig N₂. Nitrogen was blown over the cake overnight to give 580 g (94% yield) of the titled compound: ¹H NMR (DMSO), 7.090-6.985 (m, 6H), 6.930-6.877 (m, 4H), 5.900 (d, 1H, J=8.301), 5.206 (d, 1H, J=8.301).

### (4S,5R)-3-((E)-2-Methyl-hex-2-enoyl)-4,5-diphenyl-oxazolidin-2-one (Alternative A)

A 20 L jacketed reactor was fitted with a reflux condenser. To the reactor was charged 1100 g (4.597 mol) of (4S,5R)-4,5-diphenyl-oxazolidin-2-one, 884 g (6.896 mol) E)-2-methyl-2-pentenoic acid, 1705 g (6.896 mol) of EEDQ, 48 g (1.149 mol) of LiCl and 16 L of EtOAc. The reaction mixture was heated to 65° C. and was held for 200 minutes. The reaction mixture was cooled to room temperature and was extracted 3× with 3.5 L aliquots of 1N HCl. The combined aqueous extracts were filtered to give a white solid. The recovered white solid was added back to the organic layer. The 20 L reactor was fitted with a distillation head and the organic layer was distilled to remove in succession: 13.5 L of EtOAc, after which 5 L of heptane was added to the reactor; 5 L of EtOAc/heptane, after which 5 L of heptane was added to the reactor; and 2.7 L of EtOAc/heptane, after which 2.7 L of heptane was added to the reactor. The contents of the reactor were cooled to 25° C. and the resulting mixture was filtered under 5 psig nitrogen while washing with 4 L of heptane. The wet cake was dried under nitrogen pressure overnight to give 1521 g of the titled compound: ¹H NMR (DMSO), 7.12-6.94 (m, 8H), 6.834 (dd, 2H, J=7.813, 1.709), 6.060 (d, 1H, J=8.057), 6.050 (td, 1H, J=7.447, 1.221), 5.795 (d, 1H, J=8.057), 2.119-2.064 (m, 2H), 1.778 (d, 3H, J=0.997), 1.394 (m, 2H), 0.874 (t, 3H, J=7.324); Anal Calc'd for C₂₂H₂₃N₁O₃: C, 75.62; H, 6.63; N, 4.01. Found: C, 75.26; H, 6.72; N, 3.95.

### (4S,5R)-3-(2-(E)-Methyl-hex-2-enoyl)-4,5-diphenyl-oxazolidin-2-one (Alternative B)

To a solution of (E)-2-methyl-2-hexenoic acid (6.0 g, 47 mmol) in 250 mL of THF at 0° C. was added 16.3 mL (117 mmol) of triethylamine, then 5.8 mL (47 mmol) of pivaloyl chloride resulting in a thick suspension. The mixture was stirred for 1 hour at 0° C. at which time 2.0 g (47 mmol) of lithium chloride was added in one portion, followed by 10.0 g (42 mmol) of (4S,5R)-4,5-diphenyl-2-oxazolidinone in four batches. Stirring was maintained throughout the solid additions. The resulting mixture was stirred for 1 hour at 0° C., then for 1 hour at ambient temperature, and was vacuum filtered through a coarse frit and concentrated. The residue was partitioned between EtOAc/water, and the organics were dried over MgSO₄ and concentrated. To the residue was added 100 mL of MTBE and the mixture warmed cautiously with swirling. The warm slurry was filtered to provide 10.5 g (64% yield) of the titled compound as a colorless solid: ¹H NMR (CDCl₃), δ 7.12 (m, 3H), 7.07 (m, 3H), 6.94 (m, 2H), 6.84 (m, 2H), 6.17 (m, 1H), 5.89 (d, J=7.8 Hz, 1H), 5.68 (d, J=7.8 Hz, 1H), 2.18 (m, 2H), 1.92 (s, 3H), 1.50 (m, 2H), 0.96 (t, J=7.6 Hz, 3H).

### (4S,5R)-3-((2R,3R)-2,3-Dimethyl-hexanoyl)-4,5-diphenyl-oxazolidin-2-one

A 22 L 4-neck round bottom flask was equipped with an addition funnel, mechanical stirrer, and nitrogen inlet. The system was purged with nitrogen for 1 hour. THF (6 L) were charged to the flask followed by 1236 g (6.01 mol) of CuBr.S(CH₃)₂ and 364 g (8.59 mol) of LiCl. The reaction was stirred for 15 minutes at ambient temperature. The solution was cooled to −35° C. and 3.96 L (11.88 mol) of a 3M solution of CH₃MgCl in THF was charged at a rate as to keep the internal temperature of the reaction mixture below −25° C. The reaction was stirred for 1 hour after the addition of CH₃MgCl was complete. (4S,5R)-3-((E)-2-Methyl-hex-2-enoyl)-4,5-diphenyl-oxazolidin-2-one (1.00 Kg, 2.86 mol) was added as a solid in one portion and the reaction was stirred at −30° C. for 4 hours. The reaction mixture was transferred over a 2 hour period into another 22 L flask equipped with a mechanical stirrer, transfer line, vacuum line, and containing 4 L of 1:1 acetic acid:THF solution cooled in an ice-water bath. The quenched solution was stirred for 30 minutes and then diluted with 4 L of 2M NH₄OH in saturated aqueous NH₄Cl and 2 L of water. The biphasic mixture was stirred for 15 minutes and the phases separated. The organic phase was washed 4× with 4 L aliquots of the 2M NH₄OH solution. No more blue color was observed in the washes or the organic phase so the organic phase was diluted with 8 L of water and the THF was distilled off until the internal temperature of the distillation pot reached 95° C. The suspension was cooled to ambient temperature and filtered. The solids were washed with 4 L of water and suction dried to give 868.2 g of an off white solid. This material was recrystallized from 2 L of 95:5 heptane:toluene with a cooling rate of 5° C. per hour to provide 317.25 g of the titled compound as a white solid: ¹H NMR (CDCl₃), 7.12-6.85 (m, 10H), 5.90 (d, 1H, J=8.06 Hz), 5.72 (d, 1H, J=7.81), 3.83-3.76 (m, 1H), 1.95-1.89 (m, 1H), 1.35-1.31 (m, 1H). 1.11 (d, 3H, J=6.84), 1.10-0.95 (m, 3H), 0.92 (d, 3H, J=6.59), 0.76 (t, 3H, J=7.20), MS (APCI), M+1=366.2.

### (2R,3R)-2,3-Dimethyl-hexanoic acid

A 12 L, 4-necked round bottom flask, equipped with a mechanical stirrer, 500 mL addition funnel, nitrogen inlet, and thermometer, was charged with 4515 mL of THF and 330.0 g of (4S,5R)-3-((2R,3R)-2,3-dimethyl-hexanoyl)-4,5-diphenyl-oxazolidin-2-one. The resulting liquid mixture (all solids dissolved) was cooled to −5° C. to 0° C. using an acetone/ice bath. A solution of 60.6 g of LiOH—H₂O in 1800 mL of deionized water was cooled to 0° C. to 5° C. and was combined with 512 g of cold 30% (wt/wt) hydrogen peroxide in a 2 L Erlenmeyer flask. The solution was kept cold using an ice/water bath. After the oxazolidinone/THF solution in the 12 L reaction flask reached −5° C. to 0° C., the addition funnel was charged with approximately one quarter of the cold LiOH/water/H₂O₂ solution. While maintaining a nitrogen sweep to minimize oxygen concentration in the reactor headspace, the LiOH-/water/H₂O₂ solution was added dropwise to the vigorously stirred oxazolidinone/THF solution at such a rate as to maintain the reaction temp at 0° C. to 5° C. The addition funnel was recharged with approximately one quarter of the cold LiOH/water/H₂O₂ solution as required until all of the solution had been added to the reaction mixture (about 40 minutes for 0.45 mol scale). After the addition was completed, the mixture was stirred at 0° C. to 5° C. for 5 hours, during which the reaction mixture changed from a homogeneous solution to white slurry. A solution of 341 g of Na₂SO₃ and 188 g of NaHSO₃ in 2998 mL of deionized water (15 wt %) was added dropwise to the reaction mixture over about a 1.5 hour period (reaction was exothermic) via the addition funnel, while maintaining the reaction temperature at 0° C. to 10° C.

Following the addition, the reaction mixture was stirred at 0° C. to 10° C. for 1 hour. The reaction mixture was tested with potassium iodide-starch test paper to ensure the absence of peroxides. The reaction mixture was charged with 2000 mL of EtOAc and was stirred 5 minutes. The phases were separated and the aqueous phase was extracted with 2000 mL of EtOAc. The combined organic extract was washed with brine (2×1500 mL). The colorless organic solution was concentrated under vacuum (35° C.-40° C.) to a “wet,” white solid. Heptane (1000 mL) was added and the slurry was concentrated under vacuum (35° C.-40° C.) to a wet, white solid. Heptane (5000 mL) was added and the slurry was maintained at 0° C. to 5° C. for 16 hours and then at −10° C. to −5° C. for 1 hour. The cold slurry was filtered through a thin pad of celite, and the filter cake was washed with 100 mL of −10° C. to −5° C. heptane. The colorless filtrate was concentrated under vacuum (40° C.-45° C.) to give 130 g of the titled compound as a pale yellow oil: ¹H NMR (400 MHz, CHLOROFORM-D), 0.89 (t, J=7.00 Hz, 3H), 0.94 (d, J=6.8 Hz, 3H), 1.13 (d, J=7.0 Hz, 3H), 1.75-1.82 (m, 1H), 2.34-2.41 (m, 1H); GC Chiral purity: 99.18% (with 0.82% diastereomer) (direct acid method). Chemical purity: 100%. Anal. Calc'd for C₈H₁₆O₂: C, 66.63; H, 11.18. Found: C, 66.15; H, 11.41.

### (4R,5R)-4,5-Dimethyl-3-oxo-octanoic acid ethyl ester (Alternative A)

A 5 L 3-neck round bottom flask, equipped with a reflux condenser, mechanical stirrer, nitrogen inlet, and thermometer, was charged with 1390 mL of dry THF and 389.3 g of potassium ethyl malonate. MgCl₂ (217.8 g) was added in three equal portions so that the internal temperature was less than 50° C. The resulting grey slurry was heated to 55° C. to 60° C. using a temperature controlled heating mantle. The mixture was stirred at 55° C. to 60° C. for 5 hours. A 2 L 3-neck round bottom flask, equipped with a 500 mL addition funnel, mechanical stirrer, nitrogen inlet, and thermometer, was charged with 680 mL of dry THF and 286.8 g of 1,1′-carbonyldiimidazole (CDI). The addition funnel was charged portion-wise with a solution of 219.9 g of (2R,3R)-2,3-dimethyl-hexanoic acid in 350 mL of dry THF. The entire dimethyl-hexanoic acid acid/THF solution was added dropwise to the stirred CDI/THF suspension at such a rate so as to control the evolution of CO₂ and to maintain the reaction at a temperature of 20° C. to 25° C. Following the addition, the reaction mixture was stirred at 20° C. to 25° C. for 1 hour, during which the slurry became a pale yellow solution. After the 5-hour reaction time, the malonate/MgCl₂ reaction mixture was cooled to 20° C. to 25° C. and the condenser was replaced with a 1 L addition funnel. The addition funnel was charged portion-wise with the dimethylhexanoic acid/CDI/THF reaction mixture. This entire reaction mixture was added dropwise to the stirred malonate/MgCl₂/THF reaction mixture over about 10 minutes. After the addition was completed, the reaction mixture was heated to 35° C. to 40° C. Some effervescence was noted. The reaction mixture was stirred at 35° C. to 40° C. for 16 hour. The reaction mixture was cooled to 20° C. to 25° C. A 12 L 3-neck round bottom flask, equipped with a mechanical stirrer and thermometer, was charged with 3060 mL of 2N aq. HCl. The reaction mixture (a grey suspension) was added portion-wise to the aq. HCl solution while maintaining an internal temperature of 20° C.-25° C. The reaction temperature was moderated with an ice/water bath; the reaction mixture pH was about 1. Following the addition, the reaction mixture was stirred at 20° C. to 25° C. for 2 hours. The reaction mixture was subsequently charged with 4000 mL of EtOAc and was stirred for 5 minutes. The phases were separated and the aqueous phase was extracted with 2000 mL of EtOAc. The combined organic extract was washed sequentially with: 1N aq. HCl (2×1500 mL); 1000 mL of water (incomplete phase separation); half saturated aq. Na₂CO₃ (2×1500 mL); 1000 mL water; and brine (2×1000 mL). (The aqueous base wash removed unreacted malonate ester-acid.) The straw colored organic solution was concentrated under vacuum (35° C.-40° C.) to give a cloudy, pale yellow oil with some white solid present. The oil was redissolved in 1500 mL of n-heptane and was filtered. The filtrate was concentrated under vacuum (40° C.-45° C.) to give 327 g of the titled compound as a pale yellow oil: ¹H NMR (400 MHz, CHLOROFORM-D), d ppm 0.82 (t, J=7.1 Hz, 3H), 0.85 (d, J=6.8 Hz, 3H), 0.99 (d, J=7.1 Hz, 3H), 1.20 (t, J=7.3 Hz, 3H), 2.42-2.49 (m, 1H), 3.39 (s, 2H), 4.12 (q, J=7.16 Hz, 3H). GC Chemical purity: 96.24%.

### (4R,5R)-4,5-Dimethyl-3-oxo-octanoic acid ethyl ester (Alternative B)

To a solution containing 2.0 g (13.9 mmol) of (2R,3R)-2,3-dimethyl-hexanoic acid in 20 mL of dichloromethane was added 2.1 g (16.6 mmol) of chloromethylene dimethyl-ammonium chloride. After stirring the resulting solution under nitrogen for 1.5 hours, the solvent was evaporated to give (2R,3R)-2,3-dimethyl-hexanoyl chloride. Butyl lithium (32.7 ml, 52.4 mmol) was added to a solution of diisopropylamine (4.9 g, 48.5 mmol) in dry THF (20 mL) under nitrogen at 0° C. and stirred for 20 minutes. The solution was cooled to −78° C. and 4.3 g (48.5 mmol) of ethyl acetate was added. The solution was stirred at that temperature for 45 minutes. (2R,3R)-2,3-Dimethyl-hexanoyl chloride in dry THF (20 mL) was slowly added to the ethyl acetate enolate at −78° C. and the resulting reaction mixture was allowed to warm to room temperature. The reaction mixture was stirred at room temperature for 2.5 hours and was cooled to 0° C. The reaction was quenched with a saturated solution of ammonium chloride and extracted into ethyl acetate. The solution was washed with brine, dried over MgSO₄ and concentrated. The resulting residue was filtered through a silica plug, eluting with 60/40 solution of hexane/ethyl acetate to afford 2.7 g (89.2% yield) of the titled compound as an oil.

### (4R,5R)-4,5-Dimethyl-3-oxo-octanoic acid ethyl ester (Alternative C)

To a solution containing 1.0 g (6.9 mmol) of (2R,3R)-2,3-dimethyl-hexanoic acid in 10 mL of dichloromethane was added 1.1 g of chloromethylene dimethyl-ammonium chloride (8.3 mmol). The resulting solution was stirred under nitrogen for 1.5 hours. The solvent was subsequently evaporated to give (2R,3R)-2,3-dimethyl-hexanoyl chloride. To a solution containing 2.5 g (14.6 mmol) of potassium monoethyl malonate in 50 mL of acetonitrile was added 1.7 g (17.3 mmol) of magnesium chloride and 1.2 g (11.4 mmol) of triethylamine. The resulting mixture was stirred at room temperature for 2.5 hours. The reaction was cooled to 0° C. and a solution of the (2R,3R)-2,3-dimethyl-hexanoyl chloride in acetonitrile (20 mL) was slowly added followed by the addition of triethylamine (0.4 g, 0.4 mmol). The reaction was heated to 40° C. and stirred at that temperature for 6 hours. The reaction mixture was cooled to 25° C., quenched with a saturated solution of ammonium chloride and extracted into ethyl acetate. The solution was washed with brine, dried over MgSO₄ and concentrated. The resulting residue was filtered through a silica plug, eluting with 60/40 solution of hexane/ethyl acetate to afford 1.3 g (87.8% yield) of the titled compound as an oil.

### (4R,5R)-3-Methoxyamino-4,5-dimethyl-(Z)-oct-2-enoic acid ethyl ester

A 2 L 3-necked round bottom flask, equipped with magnetic stirring and nitrogen inlet, was charged with 153 g (0.71 mol) of (4R,5R)-4,5-dimethyl-3-oxo-octanoic acid ethyl ester and 600 mL of anhydrous EtOH. The solution was cooled to 0° C.-5° C. with an ice bath and 65.6 g (0.79 mol) of methoxylamine hydrochloride was added, followed by 58.6 g (0.71 mol) of sodium acetate. This flask contents were slowly warmed to room temperature (about 2 hours) and the reaction mixture was stirred at room temperature for another 24 hours. The solvent (EtOH) was removed under reduced pressure and the mixture was charged with CH₂Cl₂ (2×300 mL), which was subsequently removed. The mixture was cooled to RT, diluted with CH₂Cl₂ (300 mL), stirred at room temperature for 0.5 hours, and filtered under 5 psig of nitrogen. The filter cake was washed with CH₂Cl₂ (150 mL). The filtrate was concentrated under vacuum (50° C.) to give 172 g (99% yield) of the titled compound as a light yellow oil: ¹H NMR (400 MHz, CHLOROFORM-D), 0.87 (t, J=3.5 Hz, 5H), 0.89 (d, J=7.2 Hz, 3H), 1.08 (d, J=7.0 Hz, 3H), 1.24 (t, J=7.2 Hz, 4H), 1.3-1.55 (m, 2H), 2.25 (m, 1H), 3.15 (q, J=19.5 Hz, 2H), 3.81 (s, 3H), 4.14 (q, J=7.0 Hz, 2H).

### (4R,5R)-3-Amino-4,5-dimethyl-(Z)-oct-2-enoic acid ethyl ester

A reactor vessel charged with 171 g of (4R,5R)-3-methoxyamino-4,5-dimethyl-(Z)-oct-2-enoic acid ethyl ester, 1600 mL of MeOH, and 65 g of Raney nickel (Ra—Ni) catalyst. The methoxyamino ester was reacted with hydrogen at 50 psig to 55 psig. During the hydrogenation, additional Ra—Ni was added at reaction times of 8 hours (20 g), 21 hours (20 g), and 37 hours (8 g). After the reaction was completed (51 hours), the Ra—Ni was filtered off and the filtrate was concentrated under reduced pressure to give 150 g (>99% yield) of the titled compound as an oil: ¹H NMR (400 MHz, CHLOROFORM-D): 0.86 (t, J=4.5 Hz, 3H), 0.88 (d, J=4.9 Hz, 3H), 1.05-1.50 (m, 6H), 1.10 (d, J=7.0 Hz, 3H), 1.24 (t, J=7.2 Hz, 3H), 1.87 (m, 1H), 3.45 (s, 2H), 4.08 (q, J=7.0 Hz, 2 1).

### (4R,5R)-3-Acetylamino-4,5-dimethyl-(Z)-oct-2-enoic acid ethyl ester

To a 1 L 3-necked round bottom flask equipped with an overhead stirrer, thermocouple, addition funnel, and nitrogen inlet, was charged 150 g (0.70 mol) of (4R,5R)-3-amino-4,5-dimethyl-(Z)-oct-2-enoic acid ethyl ester and 50 mL of dry CH₂Cl₂. The reaction mixture was cooled to −20° C. To the mixture was added, successively, acetyl chloride (60 mL, 0.84 mol) and pyridine (66.8 g, 0.84 mol) over 0.5-hour time intervals. After the additions, the mixture was stirred at −20° C. to 0° C. for 2 hours and then filtered to remove the pyridine-HCl salt. The filtrate was diluted with 200 mL of CH₂Cl₂ and washed 2× with aliquots of aq NH₄Cl. The organic solution was treated with silica gel (50 g), MgSO₄ (20 g) and charcoal (20 g), and stirred at room temperature for 0.5 hours. The solids were filtered off and the filtrate was concentrated under reduced pressure to give 166.5 g (93% yield) of the titled compound as an oil: ¹H NMR (400 MHz, CHLOROFORM-D), 0.85 (t, J=7.4 Hz, 3H), 0.95 (d, J=6.8 Hz, 3H), 1.00 (d, J=7.0 Hz, 3H), 1.11 (m, 1H), 1.29 (t, J=5.8 Hz, 3H), 1.40-1.25 (m, 2H), 1.65 (m, 1H), 2.13 (s, 3H), 3.80 (m, 1H), 4.2-4.14 (m, 3H), 5.01 (s, 1H), 11.28 (s, 1H).

### (3R,4R,5R)-3-Acetylamino-4,5-dimethyl-octanoic acid ethyl ester

A reactor was charged with 166 g of (4R,5R)-3-acetylamino-4,5-dimethyl-(Z)-oct-2-enoic acid ethyl ester (substrate), 2650 mL of MeOH, and 36 g of Pd/SrCO₃ (lot#D25N17) catalyst. The substrate was reacted with H₂ at a pressure of 50 psig to 51 psig of. During hydrogenation, additional catalyst was added at a reaction time of 67 hours (10 g). After the reaction was completed (90 hours), Pd/SrCO₃ was filtered off and the filtrate was concentrated under reduced pressure to give 167 g (>99% yield) of the titled compound as an oil: ¹H NMR (400 MHz, CHLOROFORM-D): 0.82 (d, J=6.8 Hz, 3H), 0.88 (t, J=7.2 Hz, 3H), 0.90 (d, J=6.6 Hz, 3H), 1.25 (t, J=7.3 Hz, 3H), 1.00-1.58 (m, 6H), 1.96 (s, 3H), 2.52 (q, J=5.2 Hz, 2H), 3.47 (s, 1H), 4.10-4.30 (m, 2H), 4.12 (t, J=7.1 Hz, 1H), 5.9(d, 1H).

### (3R,4R,5R)-3-Amino-4,5-dimethyl-octanoic acid hydrochloride

Under nitrogen, 167 g of crude (3R,4R,5R)-3-acetylamino-4,5-dimethyl-octanoic acid ethyl ester was diluted 1100 mL of 6N HCl, stirred at room temperature for 16 hours, and then heated to reflux for another 24 hours. The reaction mixture was concentrated and recharged with 500 mL of isopropyl alcohol (IPA), which was subsequently removed. Acetonitrile (500 mL) was added to the crude white HCl salt and the mixture stirred at 20° C. to 25° C. for 1 hour. The resulting slurry was filtered, and the solids isolated to give 97 g of the titled compound (67% yield, 89.7% chemical purity; 90.7% chiral purity with two major diastereomers, 6.8% and 1.5%): ¹H NMR (CD₃OD): δ0.89 t, J=7.0 Hz, 3H), 0.94 t, J=6.9 Hz, 6H), 1.65-1.0 (m, 4H), 2.61 (dd, J=7.6 Hz, 1H), 2.73 (dd, J=4.6 HZ, 1H), 3.27 (m, J=1.6 Hz, 2H), 3.56 (m, 1H), 4.82 (s, 3H).

### (3R,4R,5R)-3-Amino-4,5-dimethyl-octanoic acid

(3R,4R,5R)-3-Amino-4,5-dimethyl-octanoic acid hydrochloride (92 g, 0.41 mol) was dissolved in 250 mL to 260 mL of dry MeOH in a 2 L 3-necked round bottom flask. To this solution was added Et₃N (0.45 mol, 45.8 g) dropwise, which formed a white precipitate. The resulting slurry was stirred at room temperature for 15 minutes. The solvent was removed to dryness. The white solid was dispersed in 1 L of CH₂Cl₂ (1 L) and stirred for 1 hour. CH₃CN (0.6 L) was added, and the slurry was stirred for another 0.5 hours. The slurry was filtered and the solids were washed 2× with 50 mL aliquots of CH₃CN, giving 71 g of the titled compound as a white solid (92% yield; 98.8% chiral purity; 99.7% chemical purity): ¹H NMR (400 MHz, CD₃OD): 0.89 (t, J=7.2 Hz, 3H), 0.91 (d, J=5.1 Hz, 3H), 0.93 (d, J=6.6 Hz, 3H), 1.02-1.65 (m, 4H), 2.26 (dd, J=10.2 Hz, 1H), 2.50 (dd, J=3.7 Hz, 1H), 3.27 (m, J=1.6 Hz, 2H), 3.33-3.28 (m, 1H), 4.82 (s, 3H).

1. A combination for the treatment of pain comprising an alpha-2-delta ligand and an atypical antipsychotic, or pharmaceutically acceptable salts thereof.

2. A combination according to claim 1 or 2, wherein the alpha-2-delta ligand is selected from gabapentin, pregabalin, [(1R,5R,6S)-6-(Aminomethyl)bicyclo[3.2.0]hept-6-yl]acetic acid, 3-(1-Aminomethyl-cyclohexylmethyl)-4H-[1,2,4]oxadiazol-5-one, C-[1-(1H-Tetrazol-5-ylmethyl)-cycloheptyl]-methylamine, (3S,4S)-(1-Aminomethyl-3,4-dimethyl-cyclopentyl)-acetic acid, (1α,3α,5α)(3-amino-methyl-bicyclo[3.2.0]hept-3-yl)-acetic acid, (3S,5R)-3-Aminomethyl-5-methyl-octanoic acid, (3S,5R)-3-amino-5-methyl-heptanoic acid, (3S,5R)-3-amino-5-methyl-nonanoic acid, (3S,5R)-3-Amino-5-methyl-octanoic acid, (2S,4S)-4-(3-chlorophenoxy)proline and (2S,4S)-4-(3-fluorobenzyl)proline, or a pharmaceutically acceptable salt thereof.

3. A combination according to claim 1 or 2, wherein the alpha-2-delta ligand is gabapentin.

4. A combination according to claim 1 or 2, wherein the alpha-2-delta ligand is pregabalin.

5. A combination according to any one of claims 1-4 wherein the atypical antipsychotic is selected from ziprasidone, olanzapine, clozapine, risperidone, sertindole, quetiapine, aripiprazole, asenapine, amisulpride, (3R,4R,5R)-3-amino-4,5-dimethyl-heptanoic acid and (3R,4R,5R)-3-amino-4,5-dimethyl-octanoic acid, or a pharmaceutically acceptable salt thereof.

6. A combination according to any one of claims 1-5 where the atypical antipsychotic is ziprasidone.

7. A pharmaceutical composition for the curative, prophylactic or palliative treatment of pain comprising a therapeutically effective amount of a combination according to any one of claims 1-6, or pharmaceutically acceptable salts thereof and a suitable carrier or excipient.

8. Use of an alpha-2-delta ligand in combination with an atypical antipsychotic, or pharmaceutically acceptable salts thereof, in the manufacture of a medicament for the curative, prophylactic or palliative treatment of pain.

9. Use according to claim 8 where the pain is neuropathic pain.

10. A method for the curative, prophylactic or palliative treatment of pain, comprising simultaneous, sequential or separate administration of a therapeutically amount of an alpha-2-delta ligand and an atypical antipsychotic, or pharmaceutically acceptable salts thereof, to a mammal in need of said treatment.

11. The method according to claim 10 where the pain is neuropathic pain.

12. A product containing and alpha-2-delta ligand and an atypical antipsychotic, or pharmaceutically acceptable salts thereof, as a combined preparation for simultaneous, separate or sequential use in the treatment of pain.
